SEQUENCE LISTING (2 sequences)

SEQ ID NO: 1 - DNA, 21 bp, Artificial Sequence
  Synthetic Polynucleotide
ttttttttttttttttttttt

SEQ ID NO: 2 - DNA, 21 bp, Artificial Sequence
  Synthetic Polynucleotide
ggtggtggtggtggtggtggt

(19) United States
(12) Patent Application Publication
(10) Pub. No.: US 2024/0309367 A1
(43) Pub. Date: Sep. 19, 2024

(54) LIPID NANOPARTICLE SPHERICAL NUCLEIC ACIDS

(71) Applicant: NORTHWESTERN UNIVERSITY, Evanston, IL (US)

(72) Inventors: Chad A. Mirkin, Wilmette, IL (US); Andrew Joseph Sinegra, Chicago, IL (US)

(21) Appl. No.: 18/271,782

(22) PCT Filed: Jan. 11, 2022
(86) PCT No.: PCT/US22/12023

§ 371 Date: Jul. 11, 2023

Related U.S. Application Data
(60) Provisional application No. 63/136,501, filed on Jan. 12, 2021.

(51) Int. Cl. C12N 15/113; A61K 9/51; A61K 31/713; A61K 47/28; A61K 47/54

(52) U.S. Cl. CPC C12N 15/113; A61K 9/513; A61K 31/713; A61K 47/28; A61K 47/549; C12N 2310/11; C12N 2310/122

(57) ABSTRACT

To treat disease, DNA and RNA therapeutics need to be delivered to target tissues and provide lasting benefit without side effects. Lipid nanoparticle spherical nucleic acids address this unmet need by using DNA and RNA sequences for nanoparticle targeting and tissue specificity. The lipid SNA structure has markedly different biodistribution properties than both lipid particles (loaded with nucleic acid) or even conventional SNAs (liposome and gold core).

### CROSS-REFERENCE TO RELATED APPLICATIONS

This application claims the priority benefit under 35 U.S.C. § 119(e) of U.S. Provisional Patent Application No. 63/136,501, filed Jan. 12, 2021, which is incorporated herein by reference in its entirety.

### STATEMENT OF GOVERNMENT INTEREST

This invention was made with government support under grant numbers CA208783, CA221747, and CA199091 awarded by the National Institutes of Health. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “2021-002_Seqlisting.txt”, which was created on Jan. 10, 2022 and is 643 bytes in size. The subject matter of the Sequence Listing is incorporated herein in its entirety by reference.

### BACKGROUND

Nucleic acids (DNA and RNA) have many potential applications in both therapeutics and diagnostics, but effective delivery at clinically relevant doses remains a challenge. The structures of these molecules create limitations with their delivery. These limitations include: rapid degradation by nucleases, poor biodistribution properties, low accumulation in target tissues, and sequestration within cellular compartments. To address these issues, many different nanoparticle carrier structures for nucleic acids have been explored.

Lipid nanoparticles are useful for facilitating intracellular delivery and cytosolic recognition of oligonucleotides and expression of messenger RNA. However, their ability to be targeted to specific tissues is thought to rely on endogenous lipid trafficking pathways (see, e.g., Akinc et al., Molecular Therapy (2010) 18:1357-1364). To try to address this limitation, extensive screening of lipid structures or sterols is often required [see, e.g., Love et al., Proceedings of the National Academy of Sciences of the United States of America (2010) 107: 1864-1869; and Patel et al., Nature Communications (2020) 983]. While these studies unveiled some relationships between nanoparticle structures and their distribution properties, there remains a need to develop a system with predictable nanoparticle targeting ability.

### SUMMARY

Numerous diseases can be treated by nucleic acid (DNA or RNA)-based therapeutics that silence harmful genes, replace missing genes, and edit mutations in a patient's DNA. However, in the body, the active sequences often do not reach the target tissues and cells. Lipid nanoparticles (LNPs) are some of the most effective carriers of nucleic acids. While LNP carriers are effective for delivery to easy-to-reach targets, often thousands of different carrier structures need to be screened to find significant enhancement in cell populations outside of the bloodstream and liver. Spherical nucleic acid (SNA) structures hasten this approach by using short DNA sequences on the surface of existing nanoparticle structures to target their delivery. The radially oriented outer sequence changes both the nanoparticle's destination in the body as well as its activity. The outer DNA sequence targets the delivery of the associated LNP, which has the nucleic acids used for gene silencing, gene replacement, or gene editing encapsulated. The outer DNA sequence provides the “address” to which the LNP will be delivered. Since DNA sequence combinations can form many different structures, this strategy provides numerous options for adding targeting, stimuli responsiveness, and diagnostic capability to LNP structures.

Applications of the technology described herein include, but are not limited to:
delivery of RNA to facilitate therapeutic protein expression
delivery of small interfering RNA (siRNA) for therapeutic gene silencing
delivery of multiple DNA and RNA strands for genome editing
delivery of DNA and RNA probes for diagnostics
delivery of DNA and RNA strands designed to modulate the immune system
co-delivery of DNA or RNA therapeutics with other pharmaceuticals
targeting delivery of encapsulated pharmaceuticals

Advantages of the technology described herein include, but are not limited to:
lipid nanoparticle (LNP) structures exist that deliver different DNA, RNA sequences
delivery to target tissues without side effects is central issue of field
two main approaches for targeting LNPs: 1.) synthesizing novel lipid structures and LNP compositions and 2.) decorating LNP surface with targeting peptides, antibodies
lipid nanoparticle spherical nucleic acids (LNP-SNAs) decorate the surface of LNPs with DNA and/or RNA sequences
DNA and RNA sequences on surface of LNP-SNAs enhance function and alter tissue targeting of the nanoparticles
This represents a highly modular platform as DNA and RNA can form many different structures and, in theory, be designed to bind any target with high specificity
DNA and RNA are useful as sensors and can be used to design therapeutic and diagnostic (theranostic) structures from lipid nanoparticles

Accordingly, in some aspects the disclosure provide a lipid nanoparticle spherical nucleic acid (LNP-SNA) comprising a lipid nanoparticle core and a shell of oligonucleotides comprised of oligonucleotides attached to the exterior of the lipid nanoparticle core, the lipid nanoparticle core comprising an encapsulated oligonucleotide, an ionizable lipid, a phospholipid, a sterol, and a lipid-polyethylene glycol (lipid-PEG) conjugate, wherein at least 10% of the oligonucleotides in the shell of oligonucleotides are covalently attached to the exterior of the lipid nanoparticle core through the lipid-PEG conjugate. In various embodiments, at least 20%, at least 30%, at least 40%, at least 50%, at least 60%, at least 70%, at least 80%, at least 90%, or at least 95% of the oligonucleotides in the shell of oligonucleotides are covalently attached to the exterior of the lipid nanoparticle core through the lipid-PEG conjugate. In some embodiments, 100% of the oligonucleotides in the shell of oligonucleotides are covalently attached to the exterior of the lipid nanoparticle core through the lipid-PEG conjugate. In some embodiments, the shell of oligonucleotides comprises about 5 to about 1000 oligonucleotides. In some embodiments, the shell of oligonucleotides comprises about 100 to about 1000 oligonucleotides. In further embodiments, the shell of oligonucleotides comprises about 400 oligonucleotides. In various embodiments, each oligonucleotide in the shell of oligonucleotides is about 5 to about 100 nucleotides in length. In further embodiments, each oligonucleotide in the shell of oligonucleotides is about 10 to about 50 nucleotides in length. In some embodiments, each oligonucleotide in the shell of oligonucleotides is about 25 nucleotides in length. In some embodiments, each oligonucleotide in the shell of oligonucleotides has the same nucleotide sequence. In some embodiments, the shell of oligonucleotides comprises at least two oligonucleotides having different nucleotide sequences. In various embodiments, the shell of oligonucleotides is comprised of single-stranded, double-stranded DNA oligonucleotides, or a combination thereof. In further embodiments, the shell of oligonucleotides is comprised of single-stranded, double-stranded RNA oligonucleotides, or a combination thereof. In some embodiments, the shell of oligonucleotides is comprised of single-stranded DNA oligonucleotides, double-stranded DNA oligonucleotides, single-stranded RNA oligonucleotides, double-stranded RNA oligonucleotides, or a combination thereof. In some embodiments, at least one oligonucleotide in the shell of oligonucleotides is a targeting oligonucleotide. In further embodiments, each oligonucleotide in the shell of oligonucleotides is a targeting oligonucleotide. In some embodiments, at least one oligonucleotide in the shell of oligonucleotides comprises or consists of a (GGT)ₙ nucleotide sequence, wherein n is 2, 3, 4, 5, 6, 7, 8, 9, 10, 11, 12, 13, 14, 15, 16, 17, 18, 19, 20, or more. In some embodiments, each oligonucleotide in the shell of oligonucleotides comprises or consists of a (GGT)ₙ nucleotide sequence, wherein n is 2, 3, 4, 5, 6, 7, 8, 9, 10, 11, 12, 13, 14, 15, 16, 17, 18, 19, 20, or more. In some embodiments, n is 7. In some embodiments, at least one oligonucleotide in the shell of oligonucleotides an aptamer. In some embodiments, at least one oligonucleotide in the shell of oligonucleotides comprises a detectable marker. In further embodiments, the shell of oligonucleotides comprises an inhibitory oligonucleotide, an immunostimulatory oligonucleotide, a gene editor substrate DNA or RNA or a combination thereof. In still further embodiments, the inhibitory oligonucleotide is an antisense oligonucleotide, small interfering RNA (siRNA), an aptamer, a short hairpin RNA (shRNA), a DNAzyme, or an aptazyme. In some embodiments, the immunostimulatory oligonucleotide is a CpG-motif containing oligonucleotide, a double-stranded DNA oligonucleotide, a double-stranded RNA oligonucleotide, or a single-stranded RNA oligonucleotide. In some embodiments, the encapsulated oligonucleotide is comprised of DNA, RNA, or a combination thereof. In further embodiments, the encapsulated oligonucleotide is an inhibitory oligonucleotide, mRNA, an immunostimulatory oligonucleotide, a mRNA encoding a gene editor protein, or a DNA or RNA gene editor substrate. In some embodiments, the inhibitory oligonucleotide is an antisense oligonucleotide, small interfering RNA (siRNA), an aptamer, a short hairpin RNA (shRNA), a DNAzyme, or an aptazyme. In further embodiments, the immunostimulatory oligonucleotide is CpG-motif containing oligonucleotide. In further embodiments, the immunostimulatory oligonucleotide is a CpG-motif containing oligonucleotide, a double-stranded DNA (dsDNA), a double-stranded RNA, or a single-stranded RNA (ssRNA). In various embodiments, the encapsulated oligonucleotide is about 5 to about 5000 nucleotides in length. In further embodiments, the encapsulated oligonucleotide is about 10 to about 4500 nucleotides in length. In still further embodiments, the encapsulated oligonucleotide is about 1500 nucleotides in length. In some embodiments, the lipid nanoparticle core comprises a plurality of encapsulated oligonucleotides. In some embodiments, at least one oligonucleotide in the plurality of encapsulated oligonucleotides comprises a detectable marker. In various embodiments, the plurality of encapsulated oligonucleotides comprises an inhibitory oligonucleotide, mRNA, an immunostimulatory oligonucleotide, mRNA encoding a gene editor protein, a DNA or RNA gene editor substrate, or a combination thereof. In further embodiments, the inhibitory oligonucleotide is an antisense oligonucleotide, small interfering RNA (siRNA), an aptamer, a short hairpin RNA (shRNA), a DNAzyme, or an aptazyme. In further embodiments, the immunostimulatory oligonucleotide is a CpG-motif containing oligonucleotide, a double-stranded DNA (dsDNA), a double-stranded RNA, or a single-stranded RNA (ssRNA).. In some embodiments, each oligonucleotide in the plurality of encapsulated oligonucleotides is about 10 to about 50 nucleotides in length. In further embodiments, each oligonucleotide in the plurality of encapsulated oligonucleotides is about 50 nucleotides in length. In some embodiments, each oligonucleotide in the plurality of encapsulated oligonucleotides has the same nucleotide sequence. In some embodiments, the plurality of encapsulated oligonucleotides comprises at least two oligonucleotides having different nucleotide sequences. In some embodiments, the ionizable lipid is dilinoleylmethyl-4-dimethylaminobutyrate (DLin-MC3-DMA), 2,2-Dilinoleyl-4-dimethylaminoethyl-[1,3]-dioxolane (DLin-KC2-DMA), C12-200, 1,2-dioleoyl-3-dimethylammonium-propane (DODAP), or a combination thereof. In some embodiments, the ionizable lipid is dilinoleylmethyl-4-dimethylaminobutyrate (DLin-MC3-DMA). In some embodiments, the LNP-SNA comprises a molar fraction of the ionizable lipid that is about 50% of the total lipid in the LNP-SNA. In further embodiments, the phospholipid is 1,2-distearoyl-sn-glycero-3-phosphocholine (DSPC), 1,2-Dihexadecanoyl phosphatidylcholine (DPPC), 1,2-dioleoyl-sn-glycero-3-phosphocholine (DOPC), 1,2-dioleoyl-sn-glycero-3-phosphoethanolamine (DOPE), or a combination thereof. In some embodiments, the phospholipid is 1,2-dioleoyl-sn-glycero-3-phosphoethanolamine (DOPE). In further embodiments, the LNP-SNA comprises a molar fraction of the phospholipid that is about 1% to about 25% of the total lipid in the LNP-SNA. In some embodiments, the LNP-SNA comprises a molar fraction of the phospholipid that is or is about 3.5% of the total lipid in the LNP-SNA. In some embodiments, the sterol is 3β-Hydroxycholest-5-ene (Cholesterol), 9,10-Secocholesta-5,7,10(19)-trien-3β-ol (Vitamin D3), 9,10-Secoergosta-5,7,10(19),22-tetraen-3β-ol (Vitamin D2), Calcipotriol, 24-Ethyl-5,22-cholestadien-3β-ol (Stigmasterol), 22,23-Dihydrostigmasterol (β-Sitosterol), 3,28-Dihydroxy-lupeol (Betulin), Lupeol, Ursolic acid, Oleanolic acid, 24α-Methylcholesterol (Campesterol), 24-Ethylcholesta-5,24(28)E-dien-3β-ol (Fucosterol), 24-Methylcholesta-5,22-dien-3β-ol (Brassicasterol), 24-Methylcholesta-5,7,22-trien-3β-ol (Ergosterol), 9,11-Dehydroergosterol, Daucosterol, or any of the foregoing sterols modified with one or more amino acids. In some embodiments, the LNP-SNA comprises a molar fraction of the sterol that is about 25% to about 45% of the total lipid in the LNP-SNA. In some embodiments, the LNP-SNA comprises a molar fraction of the sterol that is or is about 45% of the total lipid in the LNP-SNA. In further embodiments, the sterol is cholesterol. In still further embodiments, the LNP-SNA comprises a molar fraction of cholesterol that is or is about 45% of the total lipid in the LNP-SNA. In some embodiments, the lipid-polyethylene glycol (lipid-PEG) conjugate comprises 2000 Dalton (Da) polyethylene glycol. In some embodiments, the lipid-polyethylene glycol (lipid-PEG) conjugate is lipid-PEG-maleimide. In further embodiments, the lipid-PEG-maleimide is 1,2-dipalmitoryl-sn-glycero-3-phosphoethanolamine (DPPE) conjugated to 2000 Da polyethylene glycol maleimide, 1,2-dimyristoyl-sn-glycero-3-phosphoethanolamine (DMPE) conjugated to 2000 Da polyethylene glycol maleimide, or a combination thereof. In some embodiments, the LNP-SNA comprises a molar fraction of the lipid-PEG conjugate that is about 1.5% to about 3.5% of the total lipid in the LNP-SNA. In some embodiments, the LNP-SNA comprises a molar fraction of the lipid-PEG conjugate that is or is about 1.5% of the total lipid in the LNP-SNA. In various embodiments, the mass ratio between the ionizable lipid and the encapsulated oligonucleotide is about 20:1 to about 5:1. In some embodiments, a LNP-SNA of the disclosure further comprises a therapeutic agent encapsulated in the lipid nanoparticle core. In further embodiments, a LNP-SNA of the disclosure further comprises a therapeutic agent attached to the exterior of the lipid nanoparticle core. In further embodiments, the therapeutic agent is an antibody or antibody fragment, a small molecule, a peptide, an antibiotic, a growth factor, a hormone, an interferon, an interleukin, an antifungal, an antiviral, a chemotherapeutic agent, or a combination thereof. In some embodiments, a LNP-SNA of the disclosure further comprises a targeting peptide, targeting antibody, or a combination thereof attached to the exterior of the lipid nanoparticle core.

In some aspects, the disclosure provides a composition comprising a plurality of the lipid nanoparticle spherical nucleic acids (LNP-SNAs) of the disclosure. In some embodiments, a composition of the disclosure further comprises a therapeutic agent.

In some aspects, the disclosure provides a method of inhibiting expression of a gene comprising the step of hybridizing a polynucleotide encoding the gene product with a lipid nanoparticle spherical nucleic acid (LNP-SNA) or composition of the disclosure, wherein hybridizing between the polynucleotide and one or more oligonucleotides in the shell of oligonucleotides occurs over a length of the polynucleotide with a degree of complementarity sufficient to inhibit expression of the gene product. In some embodiments, expression of the gene product is inhibited in vivo. In some embodiments, expression of the gene product is inhibited in vitro.

In some aspects, the disclosure provides a method for up-regulating activity of a toll-like receptor (TLR), comprising contacting a cell having the toll-like receptor with a lipid nanoparticle spherical nucleic acid (LNP-SNA) or composition of the disclosure. In some embodiments, the shell of oligonucleotide comprises one or more oligonucleotides that is a TLR agonist. In further embodiments, the toll-like receptor is toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, toll-like receptor 13, or a combination thereof. In some embodiments, the method is performed in vitro. In some embodiments, the method is performed in vivo.

In some aspects, the disclosure provides a method for down-regulating activity of a toll-like receptor (TLR), comprising contacting a cell having the toll-like receptor with a lipid nanoparticle spherical nucleic acid (LNP-SNA) or composition of the disclosure. In some embodiments, the shell of oligonucleotides comprises one or more oligonucleotides that is a TLR antagonist. In further embodiments, the toll-like receptor is toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, toll-like receptor 13, or a combination thereof. In some embodiments, the method is performed in vitro. In some embodiments, the method is performed in vivo.

In some aspects, the disclosure provides a method of treating a disorder comprising administering an effective amount of a lipid nanoparticle spherical nucleic acid (LNP-SNA) or composition of the disclosure to a subject in need thereof, wherein the administering treats the disorder. In some embodiments, the disorder is cancer, an infectious disease, an autoimmune disease, or a combination thereof.

### BRIEF DESCRIPTION OF THE DRAWINGS

FIG. 1 depicts the synthesis of LNP-SNAs. LNPs loaded with nucleic acids are formed via the ethanol dilution method. DNA or RNA dissolved in a 10 mM sodium citrate buffer at pH 4.0 is mixed into lipids and cholesterol in ethanol at a 3:1 volume ratio. Next, the LNPs, which contain lipid-PEG-maleimides (red circles), are mixed with 3′-SH DNA (blue) for 2 hours at room temperature resulting in LNP-SNAs.

FIG. 2 shows the characterization of LNP-SNA with encapsulated mRNA. (A) Plot of average of three NanoSight runs of LNP-SNA comprised of 3.5% DOPE, 45% cholesterol, 50% D-Lin-MC3-DMa, and 1.5% DMPE-PEG(2000)-Maleimide with encapsulated Luc2 mRNA. (B) Cryo-TEM image of the same SNA.

FIG. 3 shows the conjugation of DNA to LNPs and confirmed LNP-SNA formation. 1% agarose gel run in TAE buffer confirmed conjugation of T21 DNA to LNPs after 2 hours shaking at room temperature. One equivalent of a T21-SH DNA sequence labeled with Cy5.5 was added to a formulation containing 3.5 mol % C14-PEG(2000)-maleimides. Presence of bands at higher MW than free Cy5.5 DNA (Lane 1), indicated that they were conjugated to the lipid-PEG.

FIG. 4 shows the activity of LNP-SNAs in cellular assays. (A) IRF3 induction of B-33 LNP-SNA, 2′3′-cGAMP, and B-33 LNP-SNA mixed with free dsDNA in a Raw 264.7 cell line (red ribbon represents 95% C.I., n=3 biologically independent replicates). (B) B-35 SNA silences Luc2 expression in a U87-MG-Luc2 cell line compared to the B-35 formulation with encapsulated siGFP control (24 hours, normalized to cell viability using CellTiter-Fluor™ assay, n=3 biologically independent replicates). (C) B-35 SNA enhances Luc2 gene silencing compared to the equivalent LNP formulation with no DNA conjugated to its surface (24 hours, normalized to untreated cell luminescence, one-tailed student's t-test, *=p<0.05, **=p<0.01, n=3 biologically independent replicates).

FIG. 5 shows mRNA delivery to major organs using LNP-SNAs in C57BL/6 mice. LNP-SNAs enhanced liver mRNA expression compared to the equivalent LNP. Luminescence was detected in harvested organs 6 hours after administration of 0.1 mg kg⁻¹ Luc mRNA via lateral tail vein. LNP-SNAs exhibited organ-specific function in the context of mRNA expression. Luminescence was detected in harvested organs 6 hours after administration of 0.1 mg kg⁻¹ Luc mRNA. (one-tailed student's t-test, *=p<0.05, each dot represents a biologically independent replicate, with 4-7 biologically independent replicates per treatment).

FIG. 6 shows that the LNP-SNA mRNA expression profile is sequence-dependent. (A) Luciferase (Luc) mRNA in liver, lungs, and spleen by treatment. Luminescence was detected in harvested organs 6 hours after administration of B-19 formulation of LNPs or LNP-SNAs at 0.1 mg kg⁻¹ by Luc mRNA. (B) LNP and T-SNA exhibit significant liver mRNA expression while G-SNA does not. (C) G-SNA exhibits mRNA expression in the spleen at levels comparable to LNP and T-SNA. (T-SNA is LNP functionalized with T21-SH DNA, G-SNA is LNP functionalized with (GGT)7-SH DNA sequence, N=4 biologically independent replicates).

FIG. 7 provides a depiction of the Cre system described in Example 2.

FIG. 8 shows that LNP-SNAs functionalized with an outer (GGT)7 DNA sequence did not cause significant liver genome editing. This was shown by liver flow cytometry data of Ai14 mice 2 days after injection of 0.3 mg kg⁻¹ Cre mRNA. (A) determination of tdTom positive cells in endothelial cells. (B) tdTom positive cells in hepatocytes (C) tdTom positive cells in liver B cells (D) tdTom positive cells in Kupffer cells.

FIG. 9 shows that LNP-SNAs functionalized with a GGT sequence caused genome editing by Cre mRNA in splenic monocytes. FIG. 9 depicts spleen flow cytometry data of Ai14 mice 2 days after injection of 0.3 mg kg¹ Cre mRNA.

FIG. 10 depicts results of experiments showing that a (GGT)7 outer sequence and DOPE helper lipid allowed for enhanced delivery of LNP-SNAs to major spleen cell types.

### DETAILED DESCRIPTION

To treat disease, DNA and RNA therapeutics need to be delivered to target tissues and provide lasting benefit without side effects. Lipid nanoparticle spherical nucleic acids address this unmet need by using DNA and RNA sequences for nanoparticle targeting and tissue specificity. The lipid SNA structure has markedly different biodistribution properties than both lipid particles (loaded with nucleic acid) or even conventional SNAs (liposome and gold core).

As used herein, a “targeting oligonucleotide” is an oligonucleotide that directs a LNP-SNA to a particular tissue and/or to a particular cell type. In some embodiments, a targeting oligonucleotide is an aptamer. Thus, in some embodiments, a LNP-SNA of the disclosure comprises an aptamer attached to the exterior of the lipid nanoparticle core, wherein the aptamer is designed to bind one or more receptors on the surface of a certain cell type. In some embodiments, a targeting oligonucleotide comprises or consists of a (GGT)ₙ nucleotide sequence, wherein n is or is at least 2, 3, 4, 5, 6, 7, 8, 9, 10, 11, 12, 13, 14, 15, 16, 17, 18, 19, 20, or more. In further embodiments, a targeting oligonucleotide comprises or consists of a (GGT), nucleotide sequence, wherein n is less than 2, 3, 4, 5, 6, 7, 8, 9, 10, 11, 12, 13, 14, 15, 16, 17, 18, 19, or 20. In further embodiments, a targeting oligonucleotide comprises or consists of a (GGT)ₙ nucleotide sequence, wherein n is 7. In some embodiments, a targeting oligonucleotide is a peptide oligonucleotide conjugate or an oligonucleotide-small molecule conjugate.

As used herein, an “immunostimulatory oligonucleotide” is an oligonucleotide that can stimulate (e.g., induce or enhance) an immune response. Typical examples of immunostimulatory oligonucleotides are CpG-motif containing oligonucleotides, single-stranded RNA oligonucleotides, double-stranded RNA oligonucleotides, and double-stranded DNA oligonucleotides. A “CpG-motif” is a cytosine-guanine dinucleotide sequence. Single-stranded RNA sequences can be recognized by toll-like receptors 8 and 9, double-stranded RNA sequences can be recognized by toll-like receptor 3, and double-stranded DNA can be recognized by toll-like receptor 3 and cyclic GMP-AMP synthase (cGAS).

The term “inhibitory oligonucleotide” refers to an oligonucleotide that reduces the production or expression of proteins, such as by interfering with translating mRNA into proteins in a ribosome or that are sufficiently complementary to either a gene or an mRNA encoding one or more of targeted proteins, that specifically bind to (hybridize with) the one or more targeted genes or mRNA thereby reducing expression or biological activity of the target protein. Inhibitory oligonucleotides include, without limitation, isolated or synthetic short hairpin RNA (shRNA or DNA), an antisense oligonucleotide (e.g., antisense RNA or DNA, chimeric antisense DNA or RNA), miRNA and miRNA mimics, small interfering RNA (siRNA), DNA or RNA inhibitors of innate immune receptors, an aptamer, a DNAzyme, or an aptazyme.

As used in this specification and the appended claims, the singular forms “a,” “an,” and “the” include plural reference unless the context clearly dictates otherwise.

The terms “polynucleotide” and “oligonucleotide” are interchangeable as used herein.

As used herein, the term “about,” when used to modify a particular value or range, generally means within 20 percent, e.g., within 10 percent, 5 percent, 4 percent, 3 percent, 2 percent, or 1 percent of the stated value or range.

Unless otherwise stated, all ranges contemplated herein include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

### Lipid Nanoparticle Spherical Nucleic Acids (LNP-SNAS)

Lipid nanoparticle spherical nucleic acids (LNP-SNAs) are comprised of a lipid nanoparticle core decorated with oligonucleotides. The lipid nanoparticle core comprises an encapsulated oligonucleotide, an ionizable lipid, a phospholipid, a sterol, and a lipid-polyethylene glycol (lipid-PEG) conjugate. Due to a combination of core and shell properties, the constructs have advantages over conventional liposomal SNAs and gold SNAs, for example and without limitation, with respect to nucleic acid delivery. The oligonucleotides of the oligonucleotide shell may be oriented in a variety of directions. In some embodiments the oligonucleotides are oriented radially outwards. The oligonucleotide shell comprises one or a plurality of oligonucleotides attached to the external surface of the lipid nanoparticle core. The spherical architecture of the oligonucleotide shell confers unique advantages over traditional nucleic acid delivery methods, including entry into nearly all cells independent of transfection agents, resistance to nuclease degradation, sequence-based function, targeting, and diagnostics.

In some aspects, the disclosure provides a lipid nanoparticle spherical nucleic acid (LNP-SNA) comprising a lipid nanoparticle core and a shell of oligonucleotides comprised of oligonucleotides attached to the exterior of the lipid nanoparticle core, the lipid nanoparticle core comprising an encapsulated oligonucleotide, an ionizable lipid, a phospholipid, a sterol, and a lipid-polyethylene glycol (lipid-PEG) conjugate, wherein at least 10% of the oligonucleotides in the shell of oligonucleotides are covalently attached to the exterior of the lipid nanoparticle core through the lipid-PEG conjugate. In various embodiments, a LNP-SNA comprises a targeting peptide, a targeting antibody, or a combination thereof attached to the exterior of the lipid nanoparticle core. Such targeting peptides are, for example and without limitation, antibody Fab fragments which bind surface markers of target cells and peptides designed to become charged in different pH environments. In various embodiments, one or more gene editing oligonucleotides are encapsulated in the lipid nanoparticle core of the LNP-SNA. Such gene editing oligonucleotides are, for example and without limitation, a messenger RNA (mRNA) encoding a gene editor protein, a DNA or RNA gene editor substrate (e.g., a guide RNA), or a combination thereof.

Thus, the lipid nanoparticle core comprises an encapsulated oligonucleotide, an ionizable lipid, a phospholipid, a sterol, and a lipid-polyethylene glycol (lipid-PEG) conjugate. In some embodiments, the ionizable lipid is dilinoleylmethyl-4-dimethylaminobutyrate (DLin-MC3-DMA), 2,2-Dilinoleyl-4-dimethylaminoethyl-[1,3]-dioxolane (DLin-KC2-DMA), C12-200, 1,2-dioleoyl-3-dimethylammonium-propane (DODAP), similar lipid/lipidoid structures, or a combination thereof. In some embodiments, the ionizable lipid is dilinoleylmethyl-4-dimethylaminobutyrate (DLin-MC3-DMA). In some embodiments, the phospholipid is 1,2-distearoyl-sn-glycero-3-phosphocholine (DSPC), 1,2-Dihexadecanoyl phosphatidylcholine (DPPC), 1,2-dioleoyl-sn-glycero-3-phosphocholine (DOPC), 1,2-dioleoyl-sn-glycero-3-phosphoethanolamine (DOPE), or a combination thereof. In any of the aspects or embodiments of the disclosure, the phospholipid is 1,2-dioleoyl-sn-glycero-3-phosphoethanolamine (DOPE). In further embodiments, the sterol is 3β-Hydroxycholest-5-ene (Cholesterol), 9,10-Secocholesta-5,7,10(19)-trien-3β-ol (Vitamin D3), 9,10-Secoergosta-5,7,10(19),22-tetraen-3β-ol (Vitamin D2), Calcipotriol, 24-Ethyl-5,22-cholestadien-3β-ol (Stigmasterol), 22,23-Dihydrostigmasterol (β-Sitosterol), 3,28-Dihydroxy-lupeol (Betulin), Lupeol, Ursolic acid, Oleanolic acid, 24α-Methylcholesterol (Campesterol), 24-Ethylcholesta-5,24(28)E-dien-3β-ol (Fucosterol), 24-Methylcholesta-5,22-dien-3β-ol (Brassicasterol), 24-Methylcholesta-5,7,22-trien-3β-ol (Ergosterol), 9,11-Dehydroergosterol, Daucosterol, or any of the foregoing sterols modified with one or more amino acids. In some embodiments, the lipid-polyethylene glycol (lipid-PEG) conjugate comprises 2000 Dalton (Da) polyethylene glycol. In further embodiments, the lipid-polyethylene glycol (lipid-PEG) conjugate is lipid-PEG-maleimide. In still further embodiments, the lipid-PEG-maleimide is 1,2-dipalmitoryl-sn-glycero-3-phosphoethanolamine (DPPE) conjugated to 2000 Da polyethylene glycol maleimide, 1,2-dimyristoyl-sn-glycero-3-phosphoethanolamine (DMPE) conjugated to 2000 Da polyethylene glycol maleimide, or a combination thereof.

LNP-SNAs can range in size from about 1 nanometer (nm) to about 500 nm, about 1 nm to about 400 nm, about 1 nm to about 300 nm, about 1 nm to about 200 nm, about 1 nm to about 150 nm, about 1 nm to about 100 nm, about 1 nm to about 90 nm, about 1 nm to about 80 nm in diameter, about 1 nm to about 70 nm in diameter, about 1 nm to about 60 nm in diameter, about 1 nm to about 50 nm in diameter, about 1 nm to about 40 nm in diameter, about 1 nm to about 30 nm in diameter, about 1 nm to about 20 nm in diameter, about 1 nm to about 10 nm, about 10 nm to about 150 nm in diameter, about 10 nm to about 140 nm in diameter, about 10 nm to about 130 nm in diameter, about 10 nm to about 120 nm in diameter, about 10 nm to about 110 nm in diameter, about 10 nm to about 100 nm in diameter, about 10 nm to about 90 nm in diameter, about 10 nm to about 80 nm in diameter, about 10 nm to about 70 nm in diameter, about 10 nm to about 60 nm in diameter, about 10 nm to about 50 nm in diameter, about 10 nm to about 40 nm in diameter, about 10 nm to about 30 nm in diameter, or about 10 nm to about 20 nm in diameter. In some embodiments, the LNP-SNA is, is at least, or is less than about 100 nm, 90 nm, 80 nm, 70 nm, 60 nm, 50 nm, 40 nm, 30 nm, 20, or 10 nm in diameter (or in mean diameter when there are a plurality of LNP-SNAs). In some embodiments, the size of the plurality of LNP-SNAs is from about 10 nm to about 150 nm (mean diameter), about 10 nm to about 140 nm in mean diameter, about 10 nm to about 130 nm in mean diameter, about 10 nm to about 120 nm in mean diameter, about 10 nm to about 110 nm in mean diameter, about 10 nm to about 100 nm in mean diameter, about 10 nm to about 90 nm in mean diameter, about 10 nm to about 80 nm in mean diameter, about 10 nm to about 70 nm in mean diameter, about 10 nm to about 60 nm in mean diameter, about 10 nm to about 50 nm in mean diameter, about 10 nm to about 40 nm in mean diameter, about 10 nm to about 30 nm in mean diameter, about 10 nm to about 20 nm in mean diameter, about 40 nm to about 150 nm in mean diameter, about 40 nm to about 100 nm in mean diameter, about 40 nm to about 90 nm in mean diameter, about 40 nm to about 80 nm in mean diameter, about 50 nm to about 200 nm in mean diameter, about 50 nm to about 150 nm in mean diameter, about 50 nm to about 100 nm in mean diameter, about 50 nm to about 90 nm in mean diameter, or about 50 nm to about 80 nm in mean diameter. In some embodiments, the diameter (or mean diameter for a plurality of LNP-SNAs) of the LNP-SNAs is from about 40 nm to about 150 nm, from about 50 to about 200 nm, or from about 40 to about 100 nm. In some embodiments, the size of the nanoparticles used in a method varies as required by their particular use or application. The variation of size is advantageously used to optimize certain physical characteristics of the LNP-SNAs, for example, the amount of surface area to which oligonucleotides may be attached as described herein. It will be understood that the foregoing diameters of LNP-SNAs can apply to the diameter of the lipid nanoparticle core itself or to the diameter of the lipid nanoparticle core and the shell of oligonucleotides attached thereto.

### Oligonucleotides

The disclosure provides lipid nanoparticle spherical nucleic acids (LNP-SNAs) comprising a lipid nanoparticle core and a shell of oligonucleotides attached to the exterior of the lipid nanoparticle core. As described herein, the lipid nanoparticle core comprises an encapsulated oligonucleotide. In any of the aspects or embodiments of the disclosure, an oligonucleotide comprises or consists of a (GGT)ₙ nucleotide sequence, wherein n is or is at least 2, 3, 4, 5, 6, 7, 8, 9, 10, 11, 12, 13, 14, 15, 16, 17, 18, 19, 20, or more. In further embodiments, a targeting oligonucleotide comprises or consists of a (GGT)ₙ nucleotide sequence, wherein n is less than 2, 3, 4, 5, 6, 7, 8, 9, 10, 11, 12, 13, 14, 15, 16, 17, 18, 19, or 20. In further embodiments, an oligonucleotide comprises or consists of a (GGT)ₙ nucleotide sequence, wherein n is 7. In various embodiments, the (GGT)ₙ sequence is on the 5′ end or the 3′ end of the oligonucleotide. In various embodiments, the (GGT)ₙ sequence is proximal or distal to the nanoparticle core. In various embodiments, the (GGT)ₙ sequence is on the end of the oligonucleotide that is attached to the nanoparticle core, the (GGT)ₙ sequence is on the end of the oligonucleotide that is not attached to the nanoparticle core, or both. The shell of oligonucleotides comprises, in various embodiments, an inhibitory oligonucleotide, an immunostimulatory oligonucleotide, a targeting oligonucleotide, or a combination thereof. Oligonucleotides of the disclosure include, in various embodiments, DNA oligonucleotides, RNA oligonucleotides, modified forms thereof, or a combination thereof. In any aspects or embodiments described herein, an oligonucleotide is single-stranded, double-stranded, or partially double-stranded. In any aspects or embodiments of the disclosure, an oligonucleotide comprises a detectable marker.

As described herein, modified forms of oligonucleotides are also contemplated by the disclosure which include those having at least one modified internucleotide linkage. In some embodiments, the oligonucleotide is all or in part a peptide nucleic acid. Other modified internucleoside linkages include at least one phosphorothioate linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. The oligonucleotide incorporated with the universal base analogues is able to function, e.g., as a probe in hybridization. Examples of universal bases include but are not limited to 5′-nitroindole-2′-deoxyriboside, 3-nitropyrrole, inosine and hypoxanthine.

The term “nucleotide” or its plural as used herein is interchangeable with modified forms as discussed herein and otherwise known in the art. The term “nucleobase” or its plural as used herein is interchangeable with modified forms as discussed herein and otherwise known in the art. Nucleotides or nucleobases comprise the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, oligonucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide”.

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In still further embodiments, oligonucleotide mimetics wherein both one or more sugar and/or one or more internucleotide linkage of the nucleotide units are replaced with “non-naturally occurring” groups. The bases of the oligonucleotide are maintained for hybridization. In some aspects, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

In still further embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligonucleotide consists of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where R^{H} is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═ (including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N═ (including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}, —O—CH₂—S—, —S—CH₂—O—, —CH₂—CH₂—S—, —O—CH₂—CH₂—S—, —S—CH₂—CH═ (including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(O CH₂CH₃)—O—, —O—PO(O CH₂CH₂S—R)—O—, —O—PO(BH₃)—O—, —O—PO(NHR^{N})—O—, —O—P(O)₂—NR^{H} H—, —NR^{H}—P(O)₂—O—, —O—P(O,NR^{H})—O—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—O—P(—O,S)—O—, —O—P(S)₂—O—, —NR^{H} P(O)₂—O—, —O—P(O,NR^{H})—O—, —O—PO(R″)—O—, —O—PO(CH₃)—O—, and —O—PO(NHR^{N})—O—, where R^{H} is selected form hydrogen and C₁₋₄-alkyl, and R″ is selected from C₁₋₆-alkyl and phenyl, are contemplated. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443.

Still other modified forms of oligonucleotides are described in detail in U.S. patent application No. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, or an RNA cleaving group. In one aspect, a modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In some aspects, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH₂—)ₙ group bridging the 2′ oxygen atom and the 4′ carbon atom wherein n is 1 or 2. LNAs and preparation thereof are described in WO 98/39352 and WO 99/14226.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In various aspects, an oligonucleotide of the disclosure (e.g., an oligonucleotide in the shell of oligonucleotides or an encapsulated oligonucleotide), or a modified form thereof, is generally about 10 nucleotides to about 100 nucleotides in length. More specifically, an oligonucleotide of the disclosure is about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, about 10 to about 90 nucleotides in length, about 10 to about 80 nucleotides in length, about 10 to about 70 nucleotides in length, about 10 to about 60 nucleotides in length, about 10 to about 50 nucleotides in length about 10 to about 45 nucleotides in length, about 10 to about 40 nucleotides in length, about 10 to about 35 nucleotides in length, about 10 to about 30 nucleotides in length, about 10 to about 25 nucleotides in length, about 10 to about 20 nucleotides in length, about 10 to about 15 nucleotides in length, about 18 to about 28 nucleotides in length, about 15 to about 26 nucleotides in length, and all oligonucleotides intermediate in length of the sizes specifically disclosed to the extent that the oligonucleotide is able to achieve the desired result. In further embodiments, an oligonucleotide of the disclosure (e.g., an encapsulated oligonucleotide) is about 5 nucleotides to about 5000 nucleotides in length. In further embodiments, an oligonucleotide of the disclosure is about 5 to about 4000 nucleotides in length, about 5 to about 3500 nucleotides in length, about 5 to about 3000 nucleotides in length, about 5 to about 2500 nucleotides in length, about 5 to about 2000 nucleotides in length, about 5 to about 1500 nucleotides in length, about 5 to about 1000 nucleotides in length, about 5 to about 900 nucleotides in length, about 5 to about 800 nucleotides in length, about 5 to about 700 nucleotides in length, about 5 to about 600 nucleotides in length, about 5 to about 500 nucleotides in length about 5 to about 450 nucleotides in length, about 5 to about 400 nucleotides in length, about 5 to about 350 nucleotides in length, about 5 to about 300 nucleotides in length, about 5 to about 250 nucleotides in length, about 5 to about 200 nucleotides in length, about 5 to about 150 nucleotides in length, about 5 to about 100 nucleotides in length, about 5 to about 90 nucleotides in length, about 5 to about 80 nucleotides in length, about 5 to about 70 nucleotides in length, about 5 to about 60 nucleotides in length, about 5 to about 50 nucleotides in length, about 5 to about 40 nucleotides in length, about 5 to about 30 nucleotides in length, about 5 to about 20 nucleotides in length, about 5 to about 10 nucleotides in length, and all oligonucleotides intermediate in length of the sizes specifically disclosed to the extent that the oligonucleotide is able to achieve the desired result. Accordingly, in various embodiments, an oligonucleotide of the disclosure is or is at least 5, 6, 7, 8,9, 10, 11, 12, 13, 14,15, 16, 17, 18,19,20,21,22,23, 24,25,26,27,28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 1200, 1500, 1700, 2000, 2500, 3000, 3500, 4000, 4500, 5000, or more nucleotides in length. In further embodiments, an oligonucleotide of the disclosure is less than 6,7,8,9, 10, 11, 12,13,14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 1500, 2000, 2500, 3000, 3500, 4000, 4500, 5000, or more nucleotides in length. In various embodiments, the shell of oligonucleotides attached to the exterior of the lipid nanoparticle core of the LNP-SNA comprises a plurality of oligonucleotides that all have the same length/sequence, while in some embodiments, the plurality of oligonucleotides comprises one or more oligonucleotide that have a different length and/or sequence relative to at least one other oligonucleotide in the plurality. In various embodiments, the lipid nanoparticle core comprises a plurality of oligonucleotides encapsulated therein that all have the same length/sequence, while in some embodiments, the lipid nanoparticle core comprises a plurality of oligonucleotides encapsulated therein comprising one or more oligonucleotide that have a different length and/or sequence relative to at least one other oligonucleotide in the plurality. For example and without limitation, in some embodiments the lipid nanoparticle core comprises a mRNA encoding a gene editing endonuclease (e.g., cas9) combined with a substrate guide RNA encapsulated therein for gene editing.

In some embodiments, the oligonucleotide (e.g., an oligonucleotide in the shell of oligonucleotides and/or an oligonucleotide encapsulated in a lipid nanoparticle) is an aptamer. Accordingly, all features and aspects of oligonucleotides described herein (e.g., length, type (DNA, RNA, modified forms thereof), optional presence of spacer) also apply to aptamers. Aptamers are oligonucleotide sequences that can be evolved to bind to various target analytes of interest. Aptamers may be single stranded, double stranded, or partially double stranded.

Methods of attaching detectable markers (e.g., fluorophores, radiolabels) and therapeutic agents (e.g., an antibody) as described herein to an oligonucleotide are known in the art.

In various aspects, a LNP-SNA of the disclosure has the ability to bind to a plurality of targets (e.g., polynucleotides, proteins). In some embodiments, a LNP-SNA further comprises one or more oligonucleotides that are inhibitory oligonucleotides as described herein. Such inhibitory oligonucleotides are, in various embodiments, present in the shell of oligonucleotide attached to the exterior of the lipid nanoparticle core, encapsulated in the lipid nanoparticle core, or both. Thus, in some embodiments, a LNP-SNA of the disclosure comprises one or more oligonucleotides having a sequence sufficiently complementary to a target polynucleotide to hybridize under the conditions being used. In some embodiments, the LNP-SNA comprises two or more oligonucleotides that are not identical, i.e., at least one of the oligonucleotides differ from at least one other oligonucleotide in that it has a different length and/or a different sequence. For example, if a specific polynucleotide is targeted, a single LNP-SNA has the ability to bind to multiple copies of the same target. In some embodiments, a single LNP-SNA has the ability to bind to different targets. Accordingly, in various aspects, a single LNP-SNA may be used in a method to inhibit expression of more than one gene product. In various embodiments, oligonucleotides are thus used to target specific polynucleotides, whether at one or more specific regions in the target polynucleotide, or over the entire length of the target polynucleotide as the need may be to effect a desired level of inhibition of gene expression.

In some embodiments, a LNP-SNA further comprises one or more oligonucleotides that are immunostimulatory oligonucleotides as described herein. Such immunostimulatory oligonucleotides are, in various embodiments, present in the shell of oligonucleotide attached to the exterior of the lipid nanoparticle core, encapsulated in the lipid nanoparticle core, or both. Accordingly, in various aspects and embodiments of the disclosure, a LNP-SNA of the disclosure possesses immunostimulatory activity, inhibition of gene expression activity, or both. The immunostimulatory oligonucleotide is, in any of the aspects or embodiments of the disclosure, a CpG-motif containing oligonucleotide. In various embodiments, the CpG-motif containing oligonucleotide is a class A CpG oligonucleotide, a class B CpG oligonucleotide, or a class C CpG oligonucleotide. A LNP-SNA, in various embodiments, comprises an inhibitory oligonucleotide, an immunostimulatory oligonucleotide, a targeting oligonucleotide, or a mixture thereof.

### Lipid Nanoparticle Surface/Encapsulated Density.

A surface density adequate to make the nanoparticles stable and the conditions necessary to obtain it for a desired combination of nanoparticles and oligonucleotides can be determined empirically. In general, oligonucleotides of the disclosure are attached to the exterior of the lipid nanoparticle core at a surface density of at least about 2 pmoles/cm². In some aspects, the surface density is about or at least about 15 pmoles/cm². Methods are also provided wherein oligonucleotides are bound to the exterior of the lipid nanoparticle core at a surface density of at least 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm2, at least about 19 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more. Alternatively, the density of oligonucleotides attached to the exterior of the lipid nanoparticle core is measured by the number of oligonucleotides attached to the LNP-SNA. With respect to the surface density of oligonucleotides attached to a LNP-SNA, it is contemplated that a LNP-SNA as described herein comprises about 1 to about 2,500, or about or about 1 to about 1,000, or about 1 to about 500 oligonucleotides attached to the exterior of the lipid nanoparticle core. In various embodiments, a LNP-SNA comprises about 10 to about 500, or about 10 to about 450, or about 10 to about 400, or about 10 to about 300, or about 10 to about 200, or about 10 to about 190, or about 10 to about 180, or about 10 to about 170, or about 10 to about 160, or about 10 to about 150, or about 10 to about 140, or about 10 to about 130, or about 10 to about 120, or about 10 to about 110, or about 10 to about 100, or 10 to about 90, or about 10 to about 80, or about 10 to about 70, or about 10 to about 60, or about 10 to about 50, or about 10 to about 40, or about 10 to about 30, or about 10 to about 20 oligonucleotides in the shell of oligonucleotides attached to the exterior of the lipid nanoparticle core. In some embodiments, a LNP-SNA comprises about 80 to about 500, or about 80 to about 400 oligonucleotides in the shell of oligonucleotides attached to the exterior of the lipid nanoparticle core. In further embodiments, a LNP-SNA comprises at least about 5, 10, 20, 30, 40, 45, 50, 55, 60, 65, 70, 75, 80, 85, 90, 95, 100, 105, 110, 115, 120, 125, 130, 135, 140, 145,150, 155, 160, 165, 170,175, 180, 185, 190, 195, 200, 250, 300, 350, 400, 450, 500, 550, 600, 650, 700, 750, 800, 850, 900, 950, or 1000 oligonucleotides in the shell of oligonucleotides attached to the exterior of the lipid nanoparticle core. In further embodiments, a LNP-SNA comprises or consists of 1, 2, 3, 4, 5, 10, 20, 30, 40, 45, 50, 55, 60, 65, 70, 75, 80, 85, 90, 95, 100, 105, 110, 115, 120, 125, 127, 130, 135, 140, 145, 150, 155, 160, 165, 170,175, 180, 185, 190, 195,200,300,400,500,600, 700, 800, 900, or 1000 oligonucleotides in the shell of oligonucleotides attached to the exterior of the lipid nanoparticle core. In still further embodiments, the shell of oligonucleotides attached to the lipid nanoparticle core of the LNP-SNA comprises 2, 3, 4, 5, 6, 7, 8, 9, 10, 11, 12, 13, 14, 15, 16, 17, 18, 19, 20, 50, 100, 200, 300, 400, 500, or more oligonucleotides. In some embodiments, the shell of oligonucleotides attached to the lipid nanoparticle core of the LNP-SNA comprises about 400 oligonucleotides. In some embodiments, the shell of oligonucleotides attached to the lipid nanoparticle core of the LNP-SNA comprises or consists of about or at least about 1, 2, 3, 4, 5, 6,7,8,9, 10, 11, 12,13,14, 15, 16, 17, 18, 19,20,21,22,23,24,25,26,27, 28, 29, 30, 31, 32, 33, 34, 35, 36, 37, 38, 39, 40, 41, 42, 43, 44,45,46,47,48,49,50, 100, 150,200,250,300, 350, 400, 450, 500, 550, 600, or more oligonucleotides. In various embodiments, about 2 to about 1000, or about 2 to about 500 oligonucleotides, or about 100 to about 1000 oligonucleotides, or about 50 to about 1000 oligonucleotides, or about 100 to about 500 oligonucleotides are attached to the external surface of a lipid nanoparticle core. In further embodiments, about 10 to about 1000, or about 10 to about 750, or about 10 to about 500, or about 10 to about 400, or about 10 to about 250, or about 10 to about 100, or about 50 to about 1000, or about 50 to about 750, or about 50 to about 500, or about 50 to about 250, or about 100 to about 1000, or about 100 to about 500, or about 2 to about 90, or about 2 to about 80, or about 2 to about 70, or about 2 to about 60, or about 2 to about 50, or about 2 to about 40, or about 2 to about 30, or about 2 to about 20, or about 2 to about 10, or about 10 to about 100, or about 10 to about 90, or about 10 to about 80, or about 10 to about 70, or about 10 to about 60, or about 10 to about 50, or about 10 to about 40, or about 10 to about 30, or about 10 to about 20, or about 20 to about 100, or about 20 to about 90, or about 20 to about 80, or about 20 to about 70, or about 20 to about 60, or about 20 to about 50, or about 20 to about 40, or about 20 to about 30 oligonucleotides are attached to the external surface of a lipid nanoparticle core. In still further embodiments, at least 1, at least 2, at least 3, at least 4, at least 5, at least 6, at least 7, at least 8, at least 9, or at least 10 oligonucleotides are attached to the exterior of the lipid nanoparticle core. With respect to the density of oligonucleotides encapsulated in the lipid nanoparticle core, it is contemplated that a LNP-SNA as described herein comprises about 1 to about 250, about 1 to about 220, about 1 to about 200, about 1 to about 150, about 1 to about 120, about 1 to about 100, about 1 to about 90, about 1 to about 80, about 1 to about 70, about 1 to about 60, about 1 to about 50, about 1 to about 40, about 1 to about 30, about 1 to about 20, about 1 to about 10, or about 1 to about 5 oligonucleotides encapsulated in the lipid nanoparticle core. In further embodiments, a LNP-SNA of the disclosure comprises about 10 to about 250, about 10 to about 220, about 10 to about 200, about 10 to about 150, about 10 to about 120, about 10 to about 100, about 10 to about 90, about 10 to about 80, about 10 to about 70, about 10 to about 60, about 10 to about 50, about 10 to about 40, about 10 to about 30, or about 10 to about 20 oligonucleotides encapsulated in the lipid nanoparticle core. In further embodiments, a LNP-SNA of the disclosure comprises or consists of about or at least about 1, 2,3,4,5,6,7,8,9, 10,11, 12, 13, 14,15,20,25,30,35, 40, 45, 50, 55, 60, 65, 70, 75, 80, 85, 90, 100, 120, 150, 170, 200, 220, 250 or more oligonucleotides encapsulated in the lipid nanoparticle core. In further embodiments, a LNP-SNA of the disclosure comprises less than 2, 3,4,5,6,7,8,9, 10,11,12, 13, 14, 15,20, 25,30,35,40,45, 50, 55, 60, 65, 70, 75, 80, 85, 90, 100, 120, 150, 170, 200, 220, or 250 oligonucleotides encapsulated in the lipid nanoparticle core.

### Spacers.

In some aspects, an oligonucleotide is attached to a lipid nanoparticle core through a spacer (and, in some embodiments, additionally through a linker). “Spacer” as used herein means a moiety that serves to increase distance between the nanoparticle and the oligonucleotide, or to increase distance between individual oligonucleotides when attached to the nanoparticle in multiple copies. Thus, spacers are contemplated being located between individual oligonucleotides in tandem, whether the oligonucleotides have the same sequence or have different sequences.

In some aspects, the spacer when present is an organic moiety. In some aspects, the spacer is a polymer, including but not limited to a water-soluble polymer, a nucleic acid, a polypeptide, an oligosaccharide, a carbohydrate, a lipid, an ethylglycol, or a combination thereof. In any of the aspects or embodiments of the disclosure, the spacer is an oligo(ethylene glycol)-based spacer. In various embodiments, an oligonucleotide comprises 1, 2, 3, 4, 5, or more spacer (e.g., Spacer-18 (hexaethyleneglycol)) moieties. In further embodiments, the spacer is an alkane-based spacer (e.g., C12). In some embodiments, the spacer is an oligonucleotide spacer (e.g., T5). An oligonucleotide spacer may have any sequence that does not interfere with the ability of the oligonucleotide to perform an intended function (e.g., stimulate an immune response or inhibit gene expression). In certain aspects, the bases of the oligonucleotide spacer are all adenylic acids, all thymidylic acids, all cytidylic acids, all guanylic acids, all uridylic acids, or all some other modified base.

In various embodiments, the length of the spacer is or is equivalent to at least about 2 nucleotides, at least about 3 nucleotides, at least about 4 nucleotides, at least about 5 nucleotides, 5-10 nucleotides, 10 nucleotides, 20 nucleotides, 10-30 nucleotides, or even greater than 30 nucleotides.

### Oligonucleotide Attachment to a Lipid Nanoparticle Core.

Oligonucleotides contemplated for use according to the disclosure include those attached to a nanoparticle core through any means (e.g., covalent or non-covalent attachment). In any of the aspects or embodiments of the disclosure an oligonucleotide is attached to the exterior of a lipid nanoparticle core via a covalent attachment of the oligonucleotide to a lipid-polyethylene glycol (lipid-PEG) conjugate. In some embodiments, 10% or at least 10% of the oligonucleotides in the shell of oligonucleotides are covalently attached to the exterior of the lipid nanoparticle core through the lipid-PEG conjugate. In further embodiments, at least 20%, 30%, 40%, 50%, 60%, 70%, 80%, 90% or 100% of the oligonucleotides in the shell of oligonucleotides are covalently attached to the exterior of the lipid nanoparticle core through the lipid-PEG conjugate. In some embodiments, 20%, 30%, 40%, 50%, 60%, 70%, 80%, 90% or 100% of the oligonucleotides in the shell of oligonucleotides are covalently attached to the exterior of the lipid nanoparticle core through the lipid-PEG conjugate. In still further embodiments, less than 20%, 30%, 40%, 50%, 60%, 70%, 80%, 90% or 100% of the oligonucleotides in the shell of oligonucleotides are covalently attached to the exterior of the lipid nanoparticle core through the lipid-PEG conjugate. In some embodiments, one or more oligonucleotides in the oligonucleotide shell is attached to the exterior of the lipid nanoparticle core through a lipid anchor group. The lipid anchor group is, in various embodiments, attached to the 5′- or 3′-end of the oligonucleotide. In various embodiments, the lipid anchor group is cholesterol or tocopherol.

Regardless of the means by which the oligonucleotide is attached to the lipid nanoparticle core, attachment in various aspects is effected through a 5′ linkage, a 3′ linkage, some type of internal linkage, or any combination of these attachments. In some embodiments, the oligonucleotide is covalently attached to a nanoparticle. In further embodiments, the oligonucleotide is non-covalently attached to a nanoparticle. In further embodiments, the oligonucleotide is attached to a nanoparticle via a combination of covalent and non-covalent linkage.

Methods of attachment are known to those of ordinary skill in the art and are described in U.S. Publication No. 2009/0209629, which is incorporated by reference herein in its entirety. Methods of attaching RNA to a nanoparticle are generally described in PCT/US2009/65822, which is incorporated by reference herein in its entirety. Methods of associating oligonucleotides with a liposomal particle are described in U.S. Patent Application Publication No. 20160310425, which is incorporated by reference herein in its entirety.

### LNP-SNA Synthesis

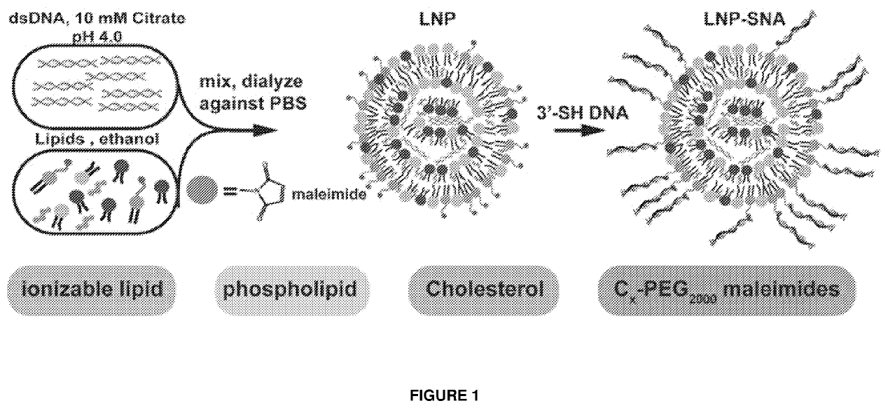

As described herein, LNP-SNAs of the disclosure generally comprise a lipid nanoparticle core comprising an encapsulated oligonucleotide and a shell of oligonucleotides attached to the exterior of the lipid nanoparticle core, wherein at least 10% of the oligonucleotides in the shell of oligonucleotides are covalently attached to the exterior of the lipid nanoparticle core through the lipid-PEG conjugate. LNP-SNAs of the disclosure are therefore synthesized such that an oligonucleotide is encapsulated in the lipid nanoparticle core and a shell of oligonucleotides is attached to the exterior of the lipid nanoparticle core. Syntheses of LNP-SNAs is described in detail herein (e.g., Example 1) and is generally depicted in FIG. 1.

In general and by way of example, lipid nanoparticles (LNPs) may be formulated by diluting the lipids and sterols in ethanol. The nucleic acids to be encapsulated are dissolved separately in a low pH (e.g., pH 4.0) buffer. The ionizable lipid to encapsulated nucleic acid mass ratios are maintained within a desired range (e.g., from 20:1 to 5:1). To form the lipid nanoparticle core, the nucleic acids in low pH buffer are rapidly mixed with the ethanol solution at a desired volume ratio (e.g., 3:1). In this process, the low pH buffer causes the ionizable lipids to become net positively charged, driving the encapsulation of the negatively charged oligonucleotides. After mixing, the nanoparticles are dialysed against 1×PBS to remove ethanol and residual buffer. Finally, to form LNP-SNAs from the LNPs, one or more oligonucleotides are attached to the exterior of the lipid nanoparticle core by mixing the oligonucleotides with the LNPs at a desired ratio (e.g., 1:1) of oligonucleotides to lipid-PEG conjugates which comprise conjugation sites.

In any of the aspects or embodiments of the disclosure, the components of the lipid nanoparticle core include an ionizable lipid, a phospholipid, a sterol, an encapsulated oligonucleotide, and a lipid-polyethylene glycol (lipid-PEG) conjugate. Various amounts of each component may be used to generate a lipid nanoparticle core. In some embodiments, the lipid nanoparticle core of the LNP-SNA comprises a molar fraction of ionizable lipid that is about 50% of the total lipid in the LNP-SNA. In some embodiments, the ionizable lipid is dilinoleylmethyl-4-dimethylaminobutyrate (DLin-MC3-DMA). In some embodiments, the lipid nanoparticle core of the LNP-SNA comprises a molar fraction of the phospholipid that is or is about 1% to about 25%, or about 2% to about 5%, or about 5% to about 20%, or about 10% to about 25%, or about 10% to about 20% of the total lipid in the LNP-SNA. In further embodiments, the lipid nanoparticle core of the LNP-SNA comprises a molar fraction of the phospholipid that is, is at least about, or is less than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% of the total lipid in the LNP-SNA. In some embodiments, the lipid nanoparticle core of the LNP-SNA comprises a molar fraction of the phospholipid that is or is about 3.5% of the total lipid in the LNP-SNA. In various embodiments, the phospholipid is 1,2-distearoyl-sn-glycero-3-phosphocholine (DSPC), 1,2-Dihexadecanoyl phosphatidylcholine (DPPC), 1,2-dioleoyl-sn-glycero-3-phosphocholine (DOPC), 1,2-dioleoyl-sn-glycero-3-phosphoethanolamine (DOPE), or a combination thereof. In some embodiments, the phospholipid is 1,2-dioleoyl-sn-glycero-3-phosphoethanolamine (DOPE). In some embodiments, the sterol is cholesterol. In some embodiments, the lipid nanoparticle core of the LNP-SNA comprises a molar fraction of the sterol that is about 20% to about 50%, or about 25% to about 45%, or about 20% to about 35%, or about 20% to about 30%, or about 25% to about 35% of the total lipid in the LNP-SNA. In further embodiments, the lipid nanoparticle core of the LNP-SNA comprises a molar fraction of the sterol that is, is at least about, or is less than about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% of the total lipid in the LNP-SNA. In some embodiments, the lipid nanoparticle core of the LNP-SNA comprises a molar fraction of the sterol that is, is at least about, or is less than about 45%. In some embodiments, the sterol is cholesterol. In some embodiments, the LNP-SNA comprises a molar fraction of cholesterol that is or is about 45% of the total lipid in the LNP-SNA. In some embodiments, the lipid-polyethylene glycol (lipid-PEG) conjugate comprises 2000 Dalton (Da) polyethylene glycol. In further embodiments, the lipid-polyethylene glycol (lipid-PEG) conjugate is lipid-PEG-maleimide. In still further embodiments, the lipid-PEG-maleimide is 1,2-dipalmitoryl-sn-glycero-3-phosphoethanolamine (DPPE) conjugated to 2000 Da polyethylene glycol maleimide, 1,2-dimyristoyl-sn-glycero-3-phosphoethanolamine (DMPE) conjugated to 2000 Da polyethylene glycol maleimide, or a combination thereof. In some embodiments, the LNP-SNA comprises a molar fraction of the lipid-PEG conjugate that is about 1% to about 5%, or about 1% to about 4%, or about 1.5% to about 5%, or about 1.5% to about 4%, or about 1% to about 3.5%, or about 1.5% to about 3%, or about 1% to about 2%, or about 1% to about 2.5% of the total lipid in the LNP-SNA. In further embodiments, the lipid nanoparticle core of the LNP-SNA comprises a molar fraction of the lipid-PEG conjugate that is, is at least about, or is less than about 1%, 1.5%, 2%, 2.5%, 3%, 3.5%, 4%, 4.5%, or 5% of the total lipid in the LNP-SNA. In some embodiments, the lipid nanoparticle core of the LNP-SNA comprises a molar fraction of the lipid-PEG conjugate that is, is at least about, or is less than about 1.5%. In some embodiments, the lipid-PEG conjugate is 1,2-dimyristoyl-sn-glycero-3-phosphoethanolamine (DMPE) conjugated to 2000 Da polyethylene glycol maleimide. In some embodiments, the mass ratio between the ionizable lipid and the encapsulated oligonucleotide in the LNP-SNA is about 5:1. In further embodiments, the mass ratio between the ionizable lipid and the encapsulated oligonucleotide in the LNP-SNA is about 20:1, 15:1, 10:1, 5:1, 4:1, 3:1, 2:1, or 1:1.

### Uses of LNP-SNAs in Gene Regulation/Therapy

It is contemplated that in any of the aspects or embodiments of the disclosure, a LNP-SNA as disclosed herein possesses the ability to regulate gene expression. Thus, in some embodiments, a LNP-SNA of the disclosure comprises a lipid nanoparticle core and a shell of oligonucleotides attached to the exterior of the lipid nanoparticle core, wherein the shell of oligonucleotides comprises one or more oligonucleotides having gene regulatory activity (e.g., inhibition of target gene expression or target cell recognition). In some embodiments, the shell of oligonucleotides attached to the exterior of the lipid nanoparticle core of the LNP-SNA comprises one or more oligonucleotides that is an inhibitory oligonucleotide as described herein. In some embodiments, an inhibitory oligonucleotide is encapsulated in the lipid nanoparticle core of the LNP-SNA. In some embodiments, an inhibitory oligonucleotide is encapsulated in the lipid nanoparticle core of the LNP-SNA and the shell of oligonucleotides attached to the exterior of the lipid nanoparticle core of the LNP-SNA comprises one or more oligonucleotides that is an inhibitory oligonucleotide. Accordingly, in some embodiments the disclosure provides methods for inhibiting gene product expression, and such methods include those wherein expression of a target gene product is inhibited by about or at least about 5%, about or at least about 10%, about or at least about 15%, about or at least about 20%, about or at least about 25%, about or at least about 30%, about or at least about 35%, about or at least about 40%, about or at least about 45%, about or at least about 50%, about or at least about 55%, about or at least about 60%, about or at least about 65%, about or at least about 70%, about or at least about 75%, about or at least about 80%, about or at least about 85%, about or at least about 90%, about or at least about 95%, about or at least about 96%, about or at least about 97%, about or at least about 98%, about or at least about 99%, or 100% compared to gene product expression in the absence of a LNP-SNA. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of LNP-SNA and a specific oligonucleotide.

Accordingly, methods of utilizing a LNP-SNA of the disclosure in gene regulation therapy are provided. This method comprises the step of hybridizing a target polynucleotide encoding the gene product with one or more oligonucleotides of a LNP-SNA that are complementary to all or a portion of the target polynucleotide, wherein hybridizing between the target polynucleotide and the oligonucleotide occurs over a length of the target polynucleotide with a degree of complementarity sufficient to inhibit expression of the gene product. The inhibition of gene expression may occur in vivo or in vitro.

In various embodiments, the inhibitory oligonucleotide utilized in the methods of the disclosure is RNA, DNA, or a modified form thereof. In various embodiments, the inhibitory oligonucleotide is antisense DNA, small interfering RNA (siRNA), an aptamer, a short hairpin RNA (shRNA), a DNAzyme, or an aptazyme.

### Uses of LNP-SNAs in Immune Regulation

Toll-like receptors (TLRs) are a class of proteins, expressed in sentinel cells, that play a key role in regulation of innate immune system. The mammalian immune system uses two general strategies to combat infectious diseases. Pathogen exposure rapidly triggers an innate immune response that is characterized by the production of immunostimulatory cytokines, chemokines and polyreactive IgM antibodies. The innate immune system is activated by exposure to Pathogen Associated Molecular Patterns (PAMPs) that are expressed by a diverse group of infectious microorganisms. The recognition of PAMPs is mediated by members of the Toll-like family of receptors. TLR receptors, such as TLR 4, TLR 8 and TLR 9 that respond to specific oligonucleotides are located inside special intracellular compartments, called endosomes. The mechanism of modulation of, for example and without limitation, TLR 4, TLR 8 and TLR 9 receptors, is based on DNA-protein interactions.

As described herein, synthetic immunostimulatory oligonucleotides that contain CpG motifs that are similar to those found in bacterial DNA stimulate a similar response of the TLR receptors. Thus, CpG oligonucleotides of the disclosure have the ability to function as TLR agonists. Other TLR agonists contemplated by the disclosure include, without limitation, single-stranded RNA and small molecules (e.g., R848 (Resiquimod)). Therefore, immunomodulatory oligonucleotides have various potential therapeutic uses, including treatment of immune deficiency and cancer. Thus, in some embodiments, a LNP-SNA of the disclosure is used in a method to modulate the activity of a toll-like receptor (TLR).

In further embodiments, a LNP-SNA of the disclosure comprises an oligonucleotide that is a TLR antagonist. In some embodiments, the TLR antagonist is a single-stranded DNA (ssDNA).

In some embodiments, down regulation of the immune system involves knocking down the gene responsible for the expression of the Toll-like receptor. This antisense approach involves use of a LNP-SNA of the disclosure to inhibit the expression of any toll-like protein.

Accordingly, in some embodiments, methods of utilizing LNP-SNAs as described herein for modulating toll-like receptors are disclosed. The method either up-regulates or down-regulates the Toll-like-receptor activity through the use of a TLR agonist or a TLR antagonist, respectively. The method comprises contacting a cell having a toll-like receptor with a LNP-SNA of the disclosure, thereby modulating the activity and/or the expression of the toll-like receptor. The toll-like receptors modulated include one or more of toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, and/or toll-like receptor 13.

### Uses of LNP-SNAs to Treat a Disorder

In some embodiments, a LNP-SNA of the disclosure is used to treat a disorder. As used herein, “treat” or “treating” means to eliminate, reduce, or ameliorating the disorder or one or more symptoms thereof. Thus, in some aspects, the disclosure provides methods of treating a disorder comprising administering an effective amount of the LNP-SNA of the disclosure to a subject (e.g., a human subject) in need thereof, wherein the administering treats the disorder. In various embodiments, the disorder is cancer, an infectious disease, an autoimmune disease, or a combination thereof. An “effective amount” of the LNP-SNA is an amount sufficient to, for example, effect gene editing, inhibit gene expression, and/or activate an innate immune response. Thus, methods of activating an innate immune response are also contemplated herein, such methods comprising administering a LNP-SNA of the disclosure to a subject in need thereof in an amount effective to activate an innate immune response in the subject.

A LNP-SNA of the disclosure can be administered via any suitable route, such as parenteral administration, intramuscular injection, subcutaneous injection, intradermal administration, and/or mucosal administration such as oral or intranasal. Additional routes of administration include but are not limited to intravenous, intraperitoneal, intranasal administration, intra-vaginal, intra-rectal, and oral administration. A combination of different routes of administration, separately or at the same time, is also contemplated by the disclosure.

### Uses of LNP-SNAs in Detection

In some embodiments, the LNP-SNAs of the disclosure are useful in nanoflare technology. The nanoflare has been previously described in the context of polynucleotide-functionalized nanoparticles for fluorescent detection of target molecule levels inside a living cell (described in U.S. Patent Application Publication No. 20100129808, incorporated herein by reference in its entirety). In this system the “flare” is detectably labeled and is, in some embodiments, one strand of a double-stranded oligonucleotide (or a portion of a single-stranded oligonucleotide) that is labeled with a detectable marker and is displaced or released from the LNP-SNA by an incoming target polynucleotide. It is thus contemplated that the nanoflare technology is useful in the context of the LNP-SNAs described herein.

### Compositions

The disclosure also provides compositions that comprise a LNP-SNA of the disclosure, or a plurality thereof. In some embodiments, the composition further comprises a pharmaceutically acceptable carrier. The term “carrier” refers to a vehicle within which the SNA as described herein is administered to a subject. Any conventional media or agent that is compatible with the LNP-SNAs according to the disclosure can be used. The term carrier encompasses diluents, excipients, adjuvants and a combination thereof.

### Therapeutic Agents

The LNP-SNAs provided herein optionally further comprise a therapeutic agent, or a plurality thereof. The therapeutic agent is, in various embodiments, simply associated with an oligonucleotide in the shell of oligonucleotides attached to the exterior of the lipid nanoparticle core of the LNP-SNA, and/or the therapeutic agent is associated with the lipid nanoparticle core of the LNP-SNA, and/or the therapeutic agent is encapsulated in the lipid nanoparticle core of the LNP-SNA. In some embodiments, the therapeutic agent is associated with the end of an oligonucleotide in the shell of oligonucleotides that is not attached to the lipid nanoparticle core (e.g., if the oligonucleotide is attached to the lipid nanoparticle core through its 3′ end, then the therapeutic agent is associated with the 5′ end of the oligonucleotide). Alternatively, in some embodiments, the therapeutic agent is associated with the end of an oligonucleotide in the shell of oligonucleotides that is attached to the lipid nanoparticle core (e.g., if the oligonucleotide is attached to the lipid nanoparticle core through its 3′ end, then the therapeutic agent is associated with the 3′ end of the oligonucleotide). In some embodiments, the therapeutic agent is covalently associated with an oligonucleotide in the shell of oligonucleotides that is attached to the exterior of the lipid nanoparticle core of the LNP-SNA. In some embodiments, the therapeutic agent is non-covalently associated with an oligonucleotide in the shell of oligonucleotides that is attached to the exterior of the lipid nanoparticle core of the LNP-SNA. However, it is understood that the disclosure provides LNP-SNAs wherein one or more therapeutic agents are both covalently and non-covalently associated with oligonucleotides in the shell of oligonucleotides that is attached to the exterior of the lipid nanoparticle core of the LNP-SNA. It will also be understood that non-covalent associations include hybridization, protein binding, and/or hydrophobic interactions. In some embodiments, a therapeutic agent is administered separately from a LNP-SNA of the disclosure. Thus, in some embodiments, a therapeutic agent is administered before, after, or concurrently with a LNP-SNA of the disclosure to treat a disorder.

Therapeutic agents contemplated by the disclosure include without limitation a protein (e.g., a therapeutic protein), a growth factor, a hormone, an interferon, an interleukin, an antibody or antibody fragment, a small molecule, a peptide, an antibiotic, an antifungal, an antiviral, a chemotherapeutic agent, or a combination thereof.

The term “small molecule,” as used herein, refers to a chemical compound or a drug, or any other low molecular weight organic compound, either natural or synthetic. By “low molecular weight” is meant compounds having a molecular weight of less than 1500 Daltons, typically between 100 and 700 Daltons.

### Detectable Markers

In any of the aspects or embodiments of the disclosure, an oligonucleotide (e.g., one or more oligonucleotides in the shell of oligonucleotides attached to the exterior of the lipid nanoparticle core of the LNP-SNA, and/or one or more oligonucleotides that are encapsulated in the lipid nanoparticle core of the LNP-SNA) comprises a detectable marker (e.g., a fluorophore and/or a radiolabel). Methods of attaching a detectable marker to an oligonucleotide or to a nanoparticle core are known in the art.

In some embodiments, a detectable marker is associated with the lipid nanoparticle core. For example and without limitation, a lipid nanoparticle core of the disclosure may be labeled with a fluorophore. In some embodiments, both the lipid nanoparticle core, one or more oligonucleotides attached to the exterior of the lipid nanoparticle core, and/or one or more oligonucleotides encapsulated within the lipid nanoparticle core comprise a fluorophore and the fluorophores may all be the same or one or more fluorophores may be different.

### REFERENCES

1.) Hajj, K. A.; Whitehead, K. A. Tools for Translation: Non-Viral Materials for Therapeutic MRNA Delivery. Nature Reviews Materials. Nature Publishing Group Sep. 12, 2017, pp 1-17.
2.) Sahin, U.; Karikó, K.; Türeci, Ō. MRNA-Based Therapeutics-Developing a New Class of Drugs. Nature Reviews Drug Discovery. Nature Publishing Group Jan. 1, 2014, pp 759-780.
3.) Rosi, N. L.; Giljohann, D. A.; Thaxton, C. S.; Lytton-Jean, A. K. R.; Han, M. S.; Mirkin, C. A. Oligonucleotide-Modified Gold Nanoparticles for Intracellular Gene Regulation. Science (80-.). 2006, 312 (5776), 1027-1030.
4.) Jensen, S. A.; Day, E. S.; Ko, C. H.; Hurley, L. A.; Luciano, J. P.; Kouri, F. M.; Merkel, T. J.; Luthi, A. J.; Patel, P. C.; Cutler, J. I.; Daniel, W. L.; Scott, A. W.; Rotz, M. W.; Meade, T. J.; Giljohann, D. A.; Mirkin, C. A.; Stegh, A. H. Spherical Nucleic Acid Nanoparticle Conjugates as an RNAi-Based Therapy for Glioblastoma. Sci. Transl. Med. 2013, 5 (209), 209ra152.
5.) Ferrer, J. R.; Sinegra, A. J.; Ivancic, D.; Yeap, X. Y.; Qiu, L.; Wang, J. J.; Zhang, Z. J.; Wertheim, J. A.; Mirkin, C. A. Structure-Dependent Biodistribution of Liposomal Spherical Nucleic Acids. ACS Nano 2020, 14 (2), 1682-1693.
6.) Banga, R. J.; Chernyak, N.; Narayan, S. P.; Nguyen, S. T.; Mirkin, C. A. Liposomal Spherical Nucleic Acids. J. Am. Chem. Soc. 2014, 136 (28), 9866-9869.
7.) Sprangers, A. J.; Hao, L.; Banga, R. J.; Mirkin, C. A. Liposomal Spherical Nucleic Acids for Regulating Long Noncoding RNAs in the Nucleus. Small 2017, 13 (10), 1602753.
8.) Maier, M. A.; Jayaraman, M.; Matsuda, S.; Liu, J.; Barros, S.; Querbes, W.; Tam, Y. K.; Ansell, S. M.; Kumar, V.; Qin, J.; Zhang, X.; Wang, Q.; Panesar, S.; Hutabarat, R.; Carioto, M.; Hettinger, J.; Kandasamy, P.; Butler, D.; Rajeev, K. G.; Pang, B.; Charisse, K.; Fitzgerald, K.; Mui, B. L.; Du, X.; Cullis, P.; Madden, T. D.; Hope, M. J.; Manoharan, M.; Akinc, A. Biodegradable Lipids Enabling Rapidly Eliminated Lipid Nanoparticles for Systemic Delivery of RNAi Therapeutics. Mol. Ther. 2013, 21 (8), 1570-1578.
9.) Semple, S. C.; Akinc, A.; Chen, J.; Sandhu, A. P.; Mui, B. L.; Cho, C. K.; Sah, D. W. Y.; Stebbing, D.; Crosley, E. J.; Yaworski, E.; Hafez, I. M.; Dorkin, J. R.; Qin, J.; Lam, K.; Rajeev, K. G.; Wong, K. F.; Jeffs, L. B.; Nechev, L.; Eisenhardt, M. L.; Jayaraman, M.; Kazem, M.; Maier, M. A.; Srinivasulu, M.; Weinstein, M. J.; Chen, Q.; Alvarez, R.; Barros, S. A.; De, S.; Klimuk, S. K.; Borland, T.; Kosovrasti, V.; Cantley, W. L.; Tam, Y. K.; Manoharan, M.; Ciufolini, M. A.; Tracy, M. A.; De Fougerolles, A.; MacLachlan, I.; Cullis, P. R.; Madden, T. D.; Hope, M. J. Rational Design of Cationic Lipids for SiRNA Delivery. Nat. Biotechnol. 2010, 28 (2), 172-176.
10.) Jayaraman, M.; Ansell, S. M.; Mui, B. L.; Tam, Y. K.; Chen, J.; Du, X.; Butler, D.; Eltepu, L.; Matsuda, S.; Narayanannair, J. K.; Rajeev, K. G.; Hafez, I. M.; Akinc, A.; Maier, M. A.; Tracy, M. A.; Cullis, P. R.; Madden, T. D.; Manoharan, M.; Hope, M. J. Maximizing the Potency of SiRNA Lipid Nanoparticles for Hepatic Gene Silencing in Vivo. Angew. Chemie—Int. Ed. 2012, 51 (34), 8529-8533.
11.) Love, K. T.; Mahon, K. P.; Levins, C. G.; Whitehead, K. A.; Querbes, W.; Dorkin, J. R.; Qin, J.; Cantley, W.; Qin, L. L.; Racie, T.; Frank-Kamenetsky, M.; Yip, K. N.; Alvarez, R.; Sah, D. W. Y.; De Fougerolles, A.; Fitzgerald, K.; Koteliansky, V.; Akinc, A.; Langer, R.; Anderson, D. G. Lipid-like Materials for Low-Dose, in Vivo Gene Silencing. Proc. Natl. Acad. Sci. U.S.A 2010, 107 (5), 1864-1869.
12.) Akinc, A.; Zumbuehl, A.; Goldberg, M.; Leshchiner, E. S.; Busini, V.; Hossain, N.; Bacallado, S. A.; Nguyen, D. N.; Fuller, J.; Alvarez, R.; Borodovsky, A.; Borland, T.; Constien, R.; De Fougerolles, A.; Dorkin, J. R.; Narayanannair Jayaprakash, K.; Jayaraman, M.; John, M.; Koteliansky, V.; Manoharan, M.; Nechev, L.; Qin, J.; Racie, T.; Raitcheva, D.; Rajeev, K. G.; Sah, D. W. Y.; Soutschek, J.; Toudjarska, I.; Vornlocher, H. P.; Zimmermann, T. S.; Langer, R.; Anderson, D. G. A Combinatorial Library of Lipid-like Materials for Delivery of RNAi Therapeutics. Nat. Biotechnol. 2008, 26 (5), 561-569.
13.) Sago, C. D.; Lokugamage, M. P.; Islam, F. Z.; Krupczak, B. R.; Sato, M.; Dahlman, J. E. Nanoparticles That Deliver RNA to Bone Marrow Identified by in Vivo Directed Evolution. J. Am. Chem. Soc. 2018, 140 (49), 17095-17105.
14.) Cheng, Q.; Wei, T.; Farbiak, L.; Johnson, L. T.; Dilliard, S. A.; Siegwart, D. J. Selective Organ Targeting (SORT) Nanoparticles for Tissue-Specific MRNA Delivery and CRISPR-Cas Gene Editing. Nat. Nanotechnol. 2020, 1-8.
15.) Chinen, A. B.; Guan, C. M.; Mirkin, C. A. Spherical Nucleic Acid Nanoparticle Conjugates Enhance G-Quadruplex Formation and Increase Serum Protein Interactions. Angew. Chemie—Int. Ed. 2015, 54 (2), 527-531.
16.) Akinc, A.; Querbes, W.; De, S.; Qin, J.; Frank-Kamenetsky, M.; Jayaprakash, K. N.; Jayaraman, M.; Rajeev, K. G.; Cantley, W. L.; Dorkin, J. R.; Butler, J. S.; Qin, L.; Racie, T.; Sprague, A.; Fava, E.; Zeigerer, A.; Hope, M. J.; Zerial, M.; Sah, D. W.; Fitzgerald, K.; Tracy, M. A.; Manoharan, M.; Koteliansky, V.; Fougerolles, A. De; Maier, M. A. Targeted Delivery of RNAi Therapeutics with Endogenous and Exogenous Ligand-Based Mechanisms. Mol. Ther. 2010, 18 (7), 1357-1364.
17.) Lokugamage, M. P.; Sago, C. D.; Gan, Z.; Krupczak, B. R.; Dahlman, J. E. Constrained Nanoparticles Deliver SiRNA and SgRNA to T Cells In Vivo without Targeting Ligands. Adv. Mater. 2019, 31 (41), 1902251.
18.) Patel, S.; Ashwanikumar, N.; Robinson, E.; Xia, Y.; Mihai, C.; Griffith Ill, J. P.; Hou, S.; Esposito, A. A.; Ketova, T.; Welsher, K.; Joyal, J. L.; Almarsson, O.; Sahay, G. Naturally occurring cholesterol analogues in lipid nanoparticles induce polymorphic shape and enhance intracellular delivery of mRNA. Nat. Comm. 2020, 11.
19.) Wang et al., Delivery of oligonucleotides with lipid nanoparticles. Advanced Drug Delivery Reviews 87: 68-80 (2015)
20.) Alfagih et al., Nanoparticles as adjuvants and nanodelivery systems for mRNA-based vaccines. Pharmaceutics 13: 45 (1-27) (2021).
21.) Veiga et al., Targeted lipid nanoparticles for RNA therapeutics and immunomodulation in leukocytes. Advanced Drug Delivery Reviews 159: 364-376 (2020).
22.) Cheng et al., Selective organ targeting (SORT) nanoparticles for tissue-specific mRNA delivery and CRISPR-Cas gene editing. Nature Nanotechnology 15: 313-320 (2020)
23.) Lokugamage et al., Constrained nanoparticles deliver siRNA and sgRNA to T cells in vivo without targeting ligands. Advanced Materials 31(41): el902251

The following examples are given merely to illustrate the present disclosure and not in any way to limit its scope.

### EXAMPLES

### Example 1

To form lipid nanoparticles (LNPs), components of the structure were dissolved in ethanol at a total concentration of 20-80 mM. These components fall under four different classes: ionizable lipid, phospholipid, sterol, and lipid-PEG-maleimide. Each nanoparticle structure was comprised of one component from each class. The molar fractions of each component were: ionizable lipid, 50%; phospholipid, 1.4-23.5%; sterol, 25-45%; lipid-PEG-maleimide, 1.5-3.5%. The ionizable lipid used was dilinoleylmethyl-4-dimethylaminobutyrate (DLin-MC3-DMA). The phospholipids used were 1,2-distearoyl-sn-glycero-3-phosphocholine (DSPC), 1,2-dioleoyl-sn-glycero-3-phosphocholine (DOPC), and 1,2-dioleoyl-sn-glycero-3-phosphoethanolamine (DOPE). The sterol used was cholesterol. The lipid-PEG-maleimides used were: 1,2-dipalmitoryl-sn-glycero-3-phosphoethanolamine (DPPE) and 1,2-dimyristoyl-sn-glycero-3-phosphoethanolamine (DMPE) conjugated to 2000 Da polyethylene glycol maleimide. The nucleic acids to be encapsulated were dissolved in 10 mM sodium citrate buffer at pH 4.0. The mass ratio used between ionizable lipid and nucleic acid used was 5:1. To encapsulate nucleic acids, the nucleic acids were mixed with the LNP components in ethanol at a volume ratio of 3:1 using a pipette tip (FIG. 1). After mixing, LNPs were dialyzed against phosphate buffered saline in 3000 Da molecular weight cutoff membranes for 2 hours.

Lipid nanoparticle spherical nucleic acids (LNP-SNAs) were formed from conjugating sulfhydryl-terminated DNA sequences to the lipid-PEG-maleimides on the surface of the dialyzed LNPs. DNA synthesized on 1-O-dimethoxytrityl-propyl-disulfide, 1′-succinyl-Icaa-CPGs was reduced to form a 3′ sulfhydryl group using 100 mM 1,4-dithiothreitol (DTT) at pH 8.3-8.5.

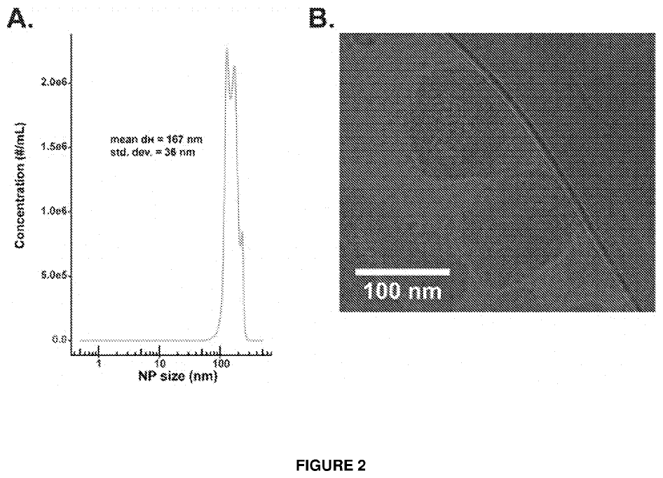

Following removal of DTT using a Sephadex G-25 column, one equivalent of DNA was mixed with LNPs, which were shaken for 2 hours at room temperature to form LNP-SNAs in phosphate buffered saline pH 7.2-7.6. The diameters and structures of LNP-SNAs were confirmed using nanoparticle tracking analysis and cryogenic electron microscopy (FIG. 2).

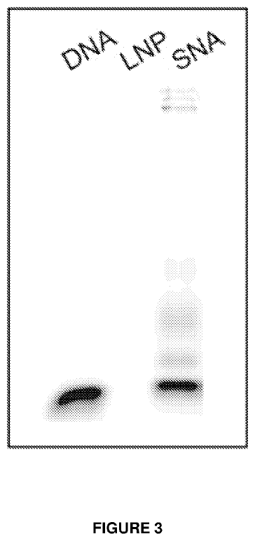

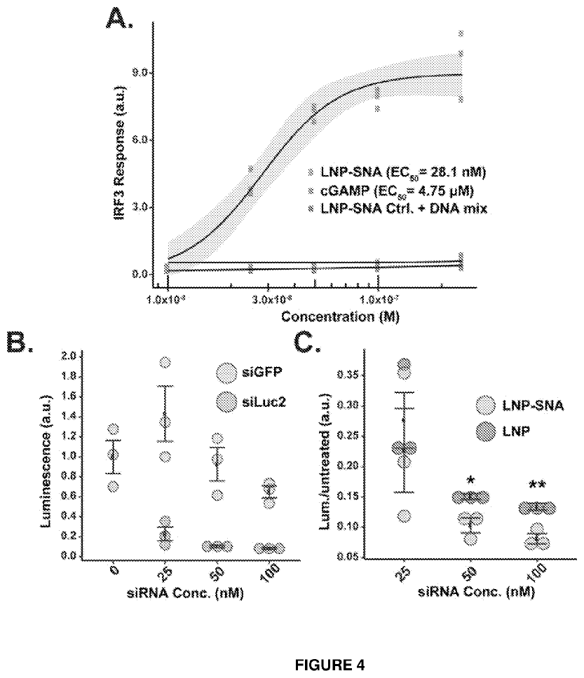

DNA mobility was reduced on an agarose gel following the reaction with LNPs, confirming conjugation (FIG. 3) LNP-SNA activity was assessed in the context of dsDNA and siRNA delivery in cellular assays. In a Raw 264.7 cell line designed to respond to activation of the cGAS-STING pathway, LNP-SNAs exhibited a critical concentration of 28.1 nM compared to 2′3′-cGAMP (4.75 μM) and free DNA, which exhibited no activation (FIG. 4A). In a U87 cell line expressing luciferase (Luc2), siLuc2 delivery was used as a proof-of-concept for LNP-SNA mediated gene silencing. LNP-SNAs containing siLuc2 silenced Luc2 expression by approximately 90% compared to siGFP control LNP-SNAs at concentrations of 25-50 nM (FIG. 4B). Compared to the equivalent LNP structure, LNP-SNAs silence Luc2 approximately 5% more at 50 and 100 nM concentrations in the U87-Luc2 cell line (FIG. 4C).

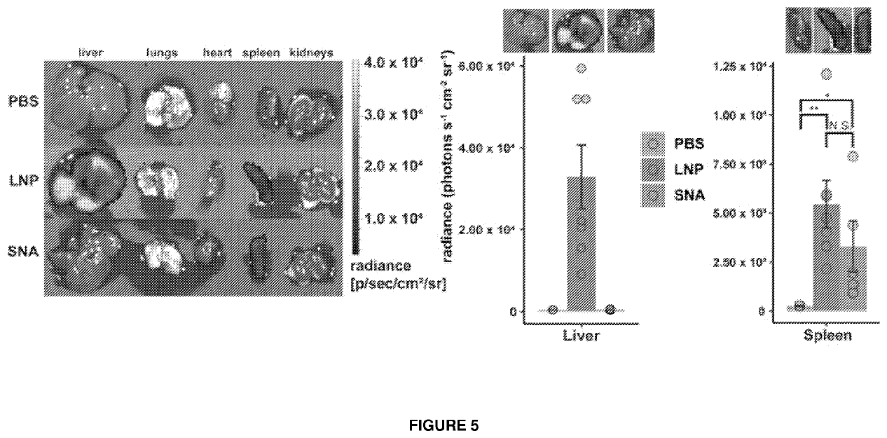

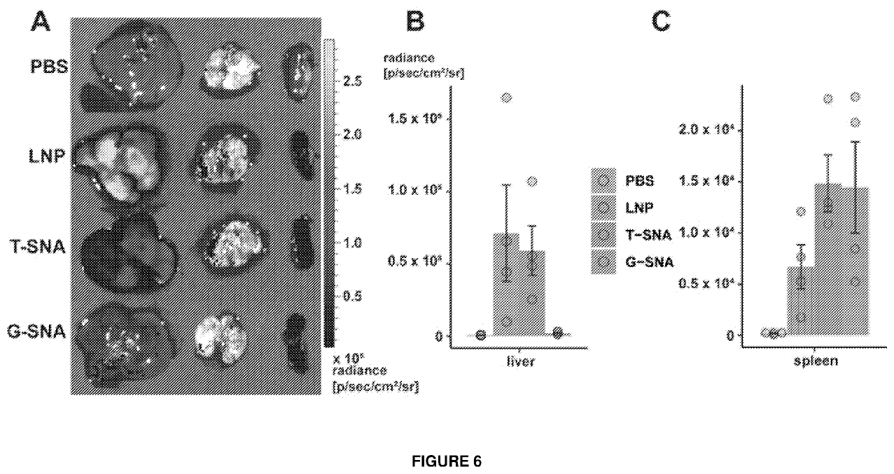

LNP-SNA function and targeting were assessed in C57BL/6 mice. LNP-SNAs and the equivalent LNPs were formulated with luciferase mRNA. After 0.1 mg kg-1 injection of mRNA via tail vein, luminescence was assessed after 6 hours. LNP-SNAs exhibited spleen-specific mRNA expression and no detectable expression in the liver (FIG. 5). In contrast, LNPs exhibited a high degree of mRNA expression in the liver, and similar level of expression in the spleen to LNP-SNAs, indicating greater potential for off-target liver toxicity. FIG. 5 shows that the existence of a DNA sequence on the surface of the SNA changes where mRNA expression is observed. In addition to altering the delivery of the equivalent LNP, delivery can be tuned by changing the sequence composition of the surface DNA. FIG. 6 demonstrates that there is a significant difference in where mRNA expression is achieved between LNP-SNAs with surface G-rich and T-rich DNA sequences.

### Materials and Methods

DNA was synthesized using automated solid support phosphoramidite synthesis (model: MM12, BioAutomation, Inc.). Sequences were purified by reverse phase high-pressure liquid chromatography (HPLC, Agilent Technologies) and characterized using matrix assisted laser desorption ionization-time of flight (MALDI-ToF, Bruker Autoflex III). The DNA sequences and lipid nanoparticle compositions used for experiments are listed in Table 1, below. Firefly luciferase mRNA was purchased from TriLink BioTechnologies.

DLin-MC3-DMA was purchased from MedChemExpress. DMPE-PEG(2000) Maleimide, DPPE-PEG(2000) Maleimide, and DSPE-PEG(2000) Maleimide were purchased from Nanocs, Inc. Cholesterol and Triton™-X-100 were purchased from Sigma. DOPC, DSPC, 18:1 DAP, and DOPE were purchased from Avanti Polar lipids, Inc. Lipofectamine™ 2000, Quant-iT™ PicoGreen™ dsDNA reagent, Quant-iT™ RiboGreen™ reagent, and 20×TE buffer were purchased from ThermoFisher. D-Luciferin was purchased from Gold Biotechnologies, and Luc mRNA was purchased from TriLink Biotechnologies.

### LNP-SNA Formulation.

LNPs were formulated using the ethanol dilution method [Cheng et al., Dendrimer-Based Lipid Nanoparticles Deliver Therapeutic FAH mRNA to Normalize Liver Function and Extend Survival in a Mouse Model of Hepatorenal Tyrosinemia Type I. Advanced Materials 30(52): el805308 (2018)]. Briefly, lipids and cholesterol were dissolved in 100% ethanol. dsDNA was dissolved in 10 mM citrate at pH 4.0 at a mass ratio of 5.0 ionizable lipid:dsDNA. After making both solutions, DNA was rapidly pipette mixed with the ethanol solution at a volume ratio of 3:1. After mixing, NPs were dialysed two times in a Pierce™ 3K MWCO microdialysis plate (ThermoFisher) for 60 min against 1×PBS. Following, NPs were added to microcentrifuge tubes containing 1 equivalent of lyophilized thiol-terminated DNA sequences and shaken at 700 rpm, room temperature, overnight to facilitate the reaction of maleimide-functionalized PEG lipids with sulfhydryl-terminated DNA.

### LNP-SNA Characterization.

LNP-SNAs size and nanoparticle concentration was determined by nanoparticle tracking analysis (NTA) using a Malvern NanoSight NS300 fitted with a NanoSight sample assistant. Nanoparticles were diluted 1:1000 in water and run through the microfluidics at 50 μL/min. Size was determined using the NTA software with a manually set detection threshold to avoid background. Encapsulation efficiency of dsDNA and RNA were determined by modified Quant-iT™ PicoGreen™ and Quant-iT™ RiboGreen™ (Invitrogen) assays respectively. Briefly, two separate standard curves were created with the encapsulated nucleic acid. One was in 1×TE Buffer while the other contained 1×TE supplemented with 0.1% Triton™-X-100. Two samples were created from each nanoparticle, one diluted in TE and one diluted in TE with 0.1% Triton™-X-100. Following, 100 μL of 1× PicoGreen™ (dsDNA) or RiboGreen™ was added on top of the standards and samples, and fluorescence of each sample was measured using a plate reader. Concentration of free nucleic acids was determined from the TE standard curve and concentration of total nucleic acids was determined by the particles lysed in 0.1% Triton™-X-100. From this, the encapsulation efficiency was calculated from the following formula: ([Triton-X]-[TE])/([Triton-X]) or ([total]-[free])/([total]).

Cellular Assays to Measure cGAS-STING Pathway Activation.

The Raw Lucia™ ISG (Raw 264.7) cell line was purchased from Invivogen. For in vitro experiments, Zeocin™ Normocin™, and QUANTI-Luc™ were also purchased from Invivogen. All cell lines were cultured according to the manufacturer's specifications. Cell line authentication was not performed. All cell lines were tested for Mycoplasma contamination and grown in a humidified atmosphere with 5% CO₂ at 37° C.

The specified nanoparticle formulations and controls were diluted in Opti-MEM (Gibco) and plated in triplicate in a 96-well plate. Following, cells were plated on top of the nanoparticle treatments at 100,000 cells per well. After 24-hour incubation, 20 μL of the media was removed and IRF3 induction was quantified using the Quanti-Luc™ reagent (Invivogen) according to the manufacturer's protocol. To normalize the number of viable cells to the amount of IRF3 induction we achieved, we used the PrestoBlue™ HS cell permeable viability reagent (Thermo Fisher). Following removing 20 μL of media for Quanti-Luc™ measurements, additional media was removed such that the volume within the plate was 90 μL. 10 μL of PrestoBlue™ was added per well and the plates were incubated for 15 minutes, at which the fluorescence was read according to the manufacturer's protocol. The IRF3 induction (luminescence) was then normalized to viable cells (PrestoBlue™ fluorescence) on a well-by-well basis.

LNP-SNAs Delivering siRNA in Cellular Assays.

B16-F10-Luc2 and U87-Luc2 cell lines were obtained from ATCC and cultured according to the manufacturer's specifications. To assess siRNA-mediated gene silencing, the top 5 LNP-SNA candidates from the cGAS-STING pathways screening were formulated with siLuc2, paired with control LNP-SNAs formulated with siGFP. Therefore, gene silencing could be read out as a decrease in luminescence due to silencing of Luc2.

The specified nanoparticle formulations and transfected siRNA controls were diluted in Opti-MEM (Gibco) and plated in triplicate in a 96-well plate. Following, cells were plated on top of the nanoparticle treatments at 50,000 cells per well. After 24-hour incubation, 120 μL of the media was removed and 20 μL of CellTiter-Fluor™ reagent (Promega) was added to measure the number of viable cells within each well. After a 30-minute incubation at 37° C., fluorescence was read according to the manufacturer's protocol. Wells were subsequently washed with 100 uL of PBS three times. Luc2 luminescence was read using the Luciferase Assay System (Promega). Luc2 gene silencing was assessed in arbitrary units normalized to the CellTiter-Fluor™ viability.

### Animal Handling.

Female mice (C57Bl/6) in the age range of 8-12 weeks were obtained from The Jackson Laboratory and maintained in conventional housing. All animals used were handled according to methods and procedures approved by the Institutional Animal Care and Use Committee at Northwestern University under protocol IS00010970.

Luciferase (Luc2) mRNA Expression.

Cleancap® Luciferase mRNA was purchased from TriLink Biotechnologies. Mice were given a single bolus injection of 0.1 mg kg⁻¹ of mRNA-containing formulations. After 6 hours, mice were injected intraperitoneally with 150 mg kg⁻¹ of D-luciferin. Following, the animals were sacrificed, and major organs were harvested and soaked in a 300 pg/mL solution of D-luciferin. Individual organs were then imaged using an IVIS Spectrum instrument (Perkin Elmer).

TABLE 1
DNA sequences and lipid nanoparticle compositions used for experiments described herein.

| | | ionizable | lipid- | surfaces | % | % | % ionizable | % lipid- | EE | size | sd |
|---|---|---|---|---|---|---|---|---|---|---|---|
| NP_# | Phospholipid | lipid | PEG | DNA | phospholipid | chol. | lipid | PEG | (%) | (nm) | (nm) |
| A_1 | DOPC | DLin-MC3-DMA | C18 | T21 | 10 | 35 | 50 | 5 | 82 | 159 | 79 |
| A_2 | DOPC | 18:1 DAP | C14 | T21 | 24 | 25 | 50 | 1 | 84 | 279 | 107 |
| A_3 | DOPC | DLin-MC3-DMA | C18 | T21 | 20 | 25 | 50 | 5 | 67 | 227 | 81 |
| A_4 | DOPC | 18:1 DAP | C14 | T21 | 14 | 35 | 50 | 1 | 85 | 221 | 105 |
| A_5 | DOPC | 18:1 DAP | C18 | T21 | 12.5 | 35 | 50 | 2.5 | 86 | 203 | 85 |
| A_6 | DOPC | DLin-MC3-DMA | C14 | T21 | 22.5 | 25 | 50 | 2.5 | 74 | 246 | 91 |
| A_7 | DOPC | DLin-MC3-DMA | C16 | T21 | 12.5 | 35 | 50 | 2.5 | 78 | 195 | 87 |
| A_8 | DOPC | 18:1 DAP | C16 | T21 | 22.5 | 25 | 50 | 2.5 | 83 | 190 | 91 |
| A_9 | DOPC | DLin-MC3-DMA | C14 | T21 | 10 | 35 | 50 | 5 | 56 | 236 | 71 |
| A_10 | DOPC | 18:1 DAP | C18 | T21 | 24 | 25 | 50 | 1 | 88 | 277 | 91 |
| A_11 | DOPC | DLin-MC3-DMA | C16 | T21 | 22.5 | 25 | 50 | 2.5 | 71 | 222 | 71 |
| A_12 | DOPC | 18:1 DAP | C16 | T21 | 12.5 | 35 | 50 | 2.5 | 87 | 178 | 83 |
| A_13 | DOPC | 18:1 DAP | C16 | T21 | 20 | 25 | 50 | 5 | 85 | 231 | 81 |
| A_14 | DOPC | DLin-MC3-DMA | C16 | T21 | 14 | 35 | 50 | 1 | 79 | 227 | 60 |
| A_15 | DOPC | 18:1 DAP | C16 | T21 | 10 | 35 | 50 | 5 | 83 | 187 | 84 |
| A_16 | DOPC | DLin-MC3-DMA | C16 | T21 | 24 | 25 | 50 | 1 | 71 | 229 | 69 |
| A_17 | DOPC | 18:1 DAP | C14 | T21 | 24 | 25 | 50 | 1 | 83 | 209 | 81 |
| A_18 | DOPC | DLin-MC3-DMA | C18 | T21 | 10 | 35 | 50 | 5 | 80 | 144 | 79 |
| B_1 | DSPC | DLin-MC3-DMA | C16 | GGT7 | 12.5 | 35 | 50 | 2.5 | 100 | 194 | 80 |
| B_2 | DSPC | DLin-MC3-DMA | C16 | GGT7 | 12.5 | 35 | 50 | 2.5 | 103 | 197 | 73 |
| B_3 | DOPE | DLin-MC3-DMA | C14 | GGT7 | 22.5 | 25 | 50 | 2.5 | 77 | 220 | 77 |
| B_4 | DOPE | DLin-MC3-DMA | C14 | GGT7 | 22.5 | 25 | 50 | 2.5 | 84 | 217 | 76 |
| B_5 | DOPC | DLin-MC3-DMA | C16 | T21 | 1.5 | 45 | 50 | 3.5 | 95 | 202 | 73 |
| B_6 | DOPC | DLin-MC3-DMA | C16 | T21 | 1.5 | 45 | 50 | 3.5 | 97 | 184 | 76 |
| B_7 | DOPE | DLin-MC3-DMA | C16 | GGT7 | 21.5 | 25 | 50 | 3.5 | 94 | 194 | 66 |
| B_8 | DSPC | DLin-MC3-DMA | C14 | T21 | 22.5 | 25 | 50 | 2.5 | 93 | 274 | 82 |
| B_9 | DOPE | DLin-MC3-DMA | C16 | GGT7 | 13.5 | 35 | 50 | 1.5 | 98 | 218 | 78 |
| B_10 | DSPC | DLin-MC3-DMA | C16 | GGT7 | 23.5 | 25 | 50 | 1.5 | 101 | 317 | 94 |
| B_11 | DOPE | DLin-MC3-DMA | C14 | T21 | 12.5 | 35 | 50 | 2.5 | 86 | 232 | 75 |
| B_12 | DOPC | DLin-MC3-DMA | C14 | GGT7 | 1.5 | 45 | 50 | 3.5 | 91 | 233 | 79 |
| B_13 | DOPE | DLin-MC3-DMA | C16 | T21 | 3.5 | 45 | 50 | 1.5 | 98 | 194 | 71 |
| B_14 | DOPC | DLin-MC3-DMA | C16 | GGT7 | 22.5 | 25 | 50 | 2.5 | 99 | 225 | 82 |
| B_15 | DSPC | DLin-MC3-DMA | C14 | T21 | 3.5 | 45 | 50 | 1.5 | 92 | 239 | 82 |
| B_16 | DSPC | DLin-MC3-DMA | C14 | T21 | 11.5 | 35 | 50 | 3.5 | 93 | 242 | 77 |
| B_17 | DSPC | DLin-MC3-DMA | C14 | GGT7 | 2.5 | 45 | 50 | 2.5 | 75 | 276 | 70 |
| B_18 | DOPE | DLin-MC3-DMA | C14 | T21 | 23.5 | 25 | 50 | 1.5 | 72 | 303 | 70 |
| B_19 | DOPE | DLin-MC3-DMA | C14 | GGT7 | 3.5 | 45 | 50 | 1.5 | 73 | 229 | 93 |
| B_20 | DOPC | DLin-MC3-DMA | C14 | GGT7 | 12.5 | 35 | 50 | 2.5 | 76 | 231 | 89 |
| B_21 | DOPC | DLin-MC3-DMA | C16 | GGT7 | 11.5 | 35 | 50 | 3.5 | 79 | 210 | 76 |
| B_22 | DSPC | DLin-MC3-DMA | C16 | T21 | 2.5 | 45 | 50 | 2.5 | 79 | 229 | 79 |
| B_23 | DOPC | DLin-MC3-DMA | C14 | T21 | 21.5 | 25 | 50 | 3.5 | 75 | 220 | 76 |
| B_24 | DSPC | DLin-MC3-DMA | C14 | GGT7 | 13.5 | 35 | 50 | 1.5 | 80 | 239 | 81 |
| B_25 | DOPC | DLin-MC3-DMA | C16 | T21 | 23.5 | 25 | 50 | 1.5 | 80 | 240 | 95 |
| B_26 | DSPC | DLin-MC3-DMA | C16 | GGT7 | 1.5 | 45 | 50 | 3.5 | 76 | 237 | 83 |
| B_27 | DOPE | DLin-MC3-DMA | C14 | T21 | 1.5 | 45 | 50 | 3.5 | 74 | 266 | 91 |
| B_28 | DSPC | DLin-MC3-DMA | C16 | T21 | 11.5 | 35 | 50 | 3.5 | 72 | 243 | 83 |
| B_29 | DOPE | DLin-MC3-DMA | C14 | GGT7 | 11.5 | 35 | 50 | 3.5 | 72 | 270 | 84 |
| B_30 | DOPC | DLin-MC3-DMA | C14 | T21 | 2.5 | 45 | 50 | 2.5 | 76 | 295 | 84 |
| B_31 | DOPE | DLin-MC3-DMA | C16 | T21 | 22.5 | 25 | 50 | 2.5 | 74 | 223 | 79 |
| B_32 | DOPC | DLin-MC3-DMA | C14 | T21 | 13.5 | 35 | 50 | 1.5 | 77 | 277 | 90 |
| B_33 | DOPC | DLin-MC3-DMA | C16 | GGT7 | 3.5 | 45 | 50 | 1.5 | 79 | 187 | 72 |
| B_34 | DOPE | DLin-MC3-DMA | C16 | GGT7 | 2.5 | 45 | 50 | 2.5 | 76 | 214 | 81 |
| B_35 | DSPC | DLin-MC3-DMA | C14 | GGT7 | 21.5 | 25 | 50 | 3.5 | 79 | 206 | 72 |
| B_36 | DOPC | DLin-MC3-DMA | C16 | T21 | 12.5 | 35 | 50 | 2.5 | 80 | 232 | 73 |
| B_37 | DOPC | DLin-MC3-DMA | C14 | GGT7 | 23.5 | 25 | 50 | 1.5 | 81 | 203 | 76 |
| C_1 | DOPC | DLin-MC3-DMA | C16 | GGT7 | 2.5 | 45 | 50 | 2.5 | 85 | 212 | 88 |
| C_2 | DOPC | DLin-MC3-DMA | C16 | GGT7 | 1.5 | 45 | 50 | 3.5 | 90 | 167 | 106 |
| C_3 | DSPC | DLin-MC3-DMA | C16 | GGT7 | 3.5 | 45 | 50 | 1.5 | 86 | 147 | 99 |
| C_4 | DOPE | DLin-MC3-DMA | C16 | GGT7 | 3.5 | 45 | 50 | 1.5 | 87 | 102 | 62 |

The percentages listed in Table 1 represent the amounts of the various components (molar percent) in the final LNP-SNA.

C14, C16 and C18 are the length of the lipid alkyl chains. The lipids are named as follows: DMPE-PEG-Maleimide (C14), DPPE-PEG-Maleimide (C16), and DSPE-PEG-Maleimide (C18). “T21” refers to the sequence 5′-TTTTTTTTTT TTTTTTTTTT T-3′ (SEQ ID NO: 1) “GGT7” refers to the sequence 5′-GGTGGTGGTG GTGGTGGTGG T-3′ (SEQ ID NO: 2) “EE %” refers to encapsulation efficiency.

### Example 2

### Ai14 Mouse Experiments

### Protocol for General Animal Handling and Isolation of Specific Cell Types

### Animal Handling.

Female mice (C57Bl/6J (#000664) and LSL-Tomato/Ai14 (#007914)) in the age range of 8-12 weeks were obtained from The Jackson Laboratory and maintained in conventional housing. All animals used were handled according to methods and procedures approved by the Institutional Animal Care and Use Committee at Northwestern University.

### Cell Isolation.

Organs were harvested and incubated in a digestion mixture of 5000 U/mL of collagenase I for 30 minutes at 37° C. Following, organs were chopped into small slices approximately 3 mm thick and pushed through 70 μm filters. Following, red blood cells were lysed using ACK lysis buffer for 5 min at RT (Thermo-Fisher) and cells were counted and resuspended in PBS containing 2.5% bovine serum albumin.

Cell types of interest derived from each organ were isolated using magnetic separation. Spleen macrophages and B-cells were isolated using EasySep™ kits (Stemcell™ Technologies). Liver B-cells were isolated using the identical EasySep™ kit. Hepatocytes were isolated via centrifugation at 200×g.

### For Ai14 Mice:

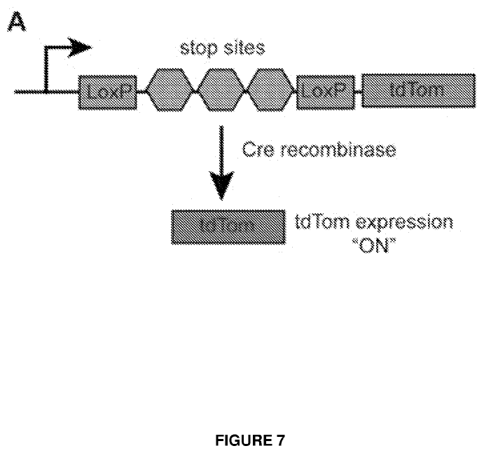

Ai14 mice were injected intravenously with LNPs and LNP-SNAs at a dose of 0.3 mg kg⁻¹ by Cre mRNA (FIG. 7). FIG. 7 depicts an assay for cell population-level genome editing using Ai14 mice. Ai14 mice express tdTom downstream of a floxed STOP cassette (FIG. 7A). Expression of Cre recombinase removes the stop cassette, turning on tdTom expression. This can be detected via flow cytometry, where the percentage of the target cell populations that express tdTom above background are quantified. This is denoted as % tdTom+ cells.

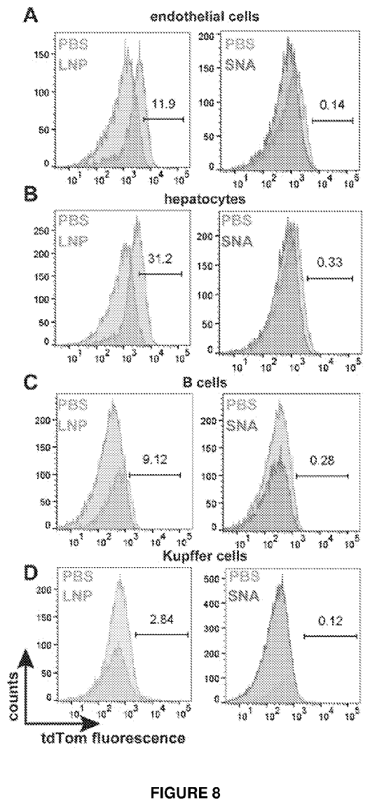

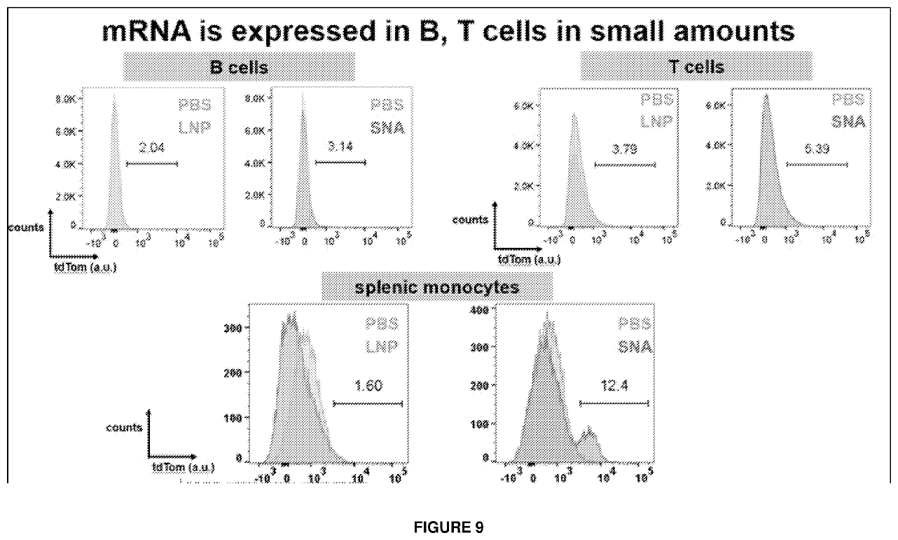

Two days after injection, animals were sacrificed and cell types of interest were isolated using the Cell isolation protocol described above. Once individual cell populations were prepared, cells were run on a flow cytometer. Gated on PBS-treated mice, genome editing in each cell type was quantified in terms of percent cells with detectable tdTomato fluorescence (FIG. 8). FIG. 9 shows that LNP-SNAs functionalized with a GGT sequence cause genome editing by Cre mRNA in splenic monocytes.

### For DNA Barcoding Experiments

### LNP-SNA Synthesis and Administration in C57BL/6J Mice.

A small library of LNP-SNAs and bare LNPs was created with the composition shown in Table 2, below. Each LNP or LNP-SNA encapsulated a unique 56-base DNA barcode identifying each particle. After quantification of the amount of barcode within each particle, an equal amount of barcode was pooled into a total dose of 0.1 mg kg⁻¹ and injected into C57BL/6J mice. After a circulation period of 2 days, cell types of interest were isolated via magnetic separation by the protocol above, and proceeded to DNA isolation and sequencing (described below).

TABLE 2
Formulations used in LNP-SNA Barcode study.

| barcode | | % | % | % D-Lin- | % PEG- | | DNA |
|---|---|---|---|---|---|---|---|
| # | phospholipid | phospholipid | chol | MC3-DMA | Mal | PEG_length | Seq. |
| 1 | DOPC | 24 | 25 | 50 | 1 | C16 | (GGT)7 |
| 2 | DOPC | 3.5 | 45 | 50 | 1.5 | C16 | (GGT)7 |
| 3 | DOPE | 2.5 | 45 | 50 | 2.5 | C16 | (GGT)7 |
| 4 | DSPC | 21.5 | 25 | 50 | 3.5 | C14 | (GGT)7 |
| 5 | DOPC | 1.5 | 45 | 50 | 3.5 | C16 | (GGT)7 |
| 6 | DOPC | 24 | 25 | 50 | 1 | C16 | NA |
| 7 | DOPC | 3.5 | 45 | 50 | 1.5 | C16 | NA |
| 8 | DOPE | 2.5 | 45 | 50 | 2.5 | C16 | NA |
| 9 | DSPC | 21.5 | 25 | 50 | 3.5 | C14 | NA |
| 10 | DOPC | 1.5 | 45 | 50 | 3.5 | C16 | NA |

### DNA Isolation, and Next-Generation Sequencing.

With cell types of interest, DNA was isolated using Clarity OTX™ columns (Phenomenex). The samples were lyophilized and cleaned using a PCR-Cleanup kit (New England Biolabs, Inc.). Following, nested PCR was performed according to previous protocols [Paunovska, K.; Sago, C. D.; Monaco, C. M.; Hudson, W. H.; Castro, M. G.; Rudoltz, T. G.; Kalathoor, S.; Vanover, D. A.; Santangelo, P. J.; Ahmed, R.; Bryksin, A. V.; Dahlman, J. E. A Direct Comparison of in Vitro and in Vivo Nucleic Acid Delivery Mediated by Hundreds of Nanoparticles Reveals a Weak Correlation. Nano Lett. 2018, 18 (3), 2148-2157. https://doi.org/10.1021/acs.nanolett.8b00432]. A universal primer was used to amplify barcode sequences, followed by adapter sequences used to index samples and add Nextera XT chemistry. Samples were sequenced using an Illumina NextSeq™

### Analysis of NGS Data.

Sequence files were analyzed using a custom R script. First, reads were preprocessed to filter out adapter primer sequences as well as reads shorter than 40 bases. Next, reads were processed so that there are no reads containing a quality score less than 20. Finally, each barcode was counted within each sample by searching for the reverse complement of the barcode sequence. With the number of reads of each barcode in each sample, the numbers were normalized to the input. This was used to normalize the number of reads from each barcode to how many were originally injected. Following, delivery was quantified as “normalized delivery” or the percent of normalized reads from each barcode as a percentage of the total number of reads in the sample.

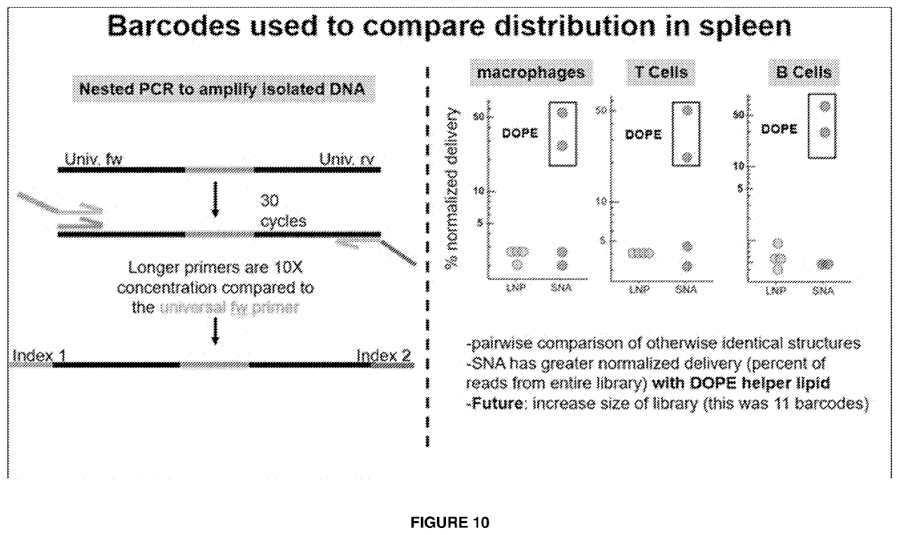

FIG. 10 shows that a (GGT)7 outer sequence and DOPE helper lipid allowed for enhanced delivery of LNP-SNAs to major spleen cell types. Delivery to the spleen was assessed using a DNA barcoding technique. LNP-SNAs were indexed with 56 nucleotide long barcodes, amplified and indexed using the strategy depicted in the left panel of FIG. 10. Delivery of LNP and LNP-SNA structures was assessed as a percent of the total barcode reads derived from each cell type of interest. Here, it was found that LNP-SNAs' enrichment in the spleen was achieved by presenting a (GGT)7 outer sequence and containing the helper lipid DOPE.

What is claimed is:

1. A lipid nanoparticle spherical nucleic acid (LNP-SNA) comprising a lipid nanoparticle core and a shell of oligonucleotides comprised of oligonucleotides attached to the exterior of the lipid nanoparticle core,
the lipid nanoparticle core comprising an encapsulated oligonucleotide, an ionizable lipid, a phospholipid, a sterol, and a lipid-polyethylene glycol (lipid-PEG) conjugate,
wherein at least 10% of the oligonucleotides in the shell of oligonucleotides are covalently attached to the exterior of the lipid nanoparticle core through the lipid-PEG conjugate.

2. The LNP-SNA of claim 1, wherein the shell of oligonucleotides comprises about 5 to about 1000 oligonucleotides.

3. The LNP-SNA of claim 1 or claim 2, wherein the shell of oligonucleotides comprises about 100 to about 1000 oligonucleotides.

4. The LNP-SNA of any one of claims 1-3, wherein the shell of oligonucleotides comprises about 400 oligonucleotides.

5. The LNP-SNA of any one of claims 1-4, wherein each oligonucleotide in the shell of oligonucleotides is about 5 to about 100 nucleotides in length.

6. The LNP-SNA of any one of claims 1-5, wherein each oligonucleotide in the shell of oligonucleotides is about 10 to about 50 nucleotides in length.

7. The LNP-SNA of any one of claims 1-6, wherein each oligonucleotide in the shell of oligonucleotides is about 25 nucleotides in length.

8. The LNP-SNA of any one of claims 1-7, wherein each oligonucleotide in the shell of oligonucleotides has the same nucleotide sequence.

9. The LNP-SNA of any one of claims 1-7, wherein the shell of oligonucleotides comprises at least two oligonucleotides having different nucleotide sequences.

10. The LNP-SNA of any one of claims 1-9, wherein the shell of oligonucleotides is comprised of single-stranded, double-stranded DNA oligonucleotides, or a combination thereof.

11. The LNP-SNA of any one of claims 1-9, wherein the shell of oligonucleotides is comprised of single-stranded, double-stranded RNA oligonucleotides, or a combination thereof.

12. The LNP-SNA of any one of claims 1-9, wherein the shell of oligonucleotides is comprised of single-stranded DNA oligonucleotides, double-stranded DNA oligonucleotides, single-stranded RNA oligonucleotides, double-stranded RNA oligonucleotides, or a combination thereof.

13. The LNP-SNA of any one of claims 1-12, wherein at least one oligonucleotide in the shell of oligonucleotides is a targeting oligonucleotide.

14. The LNP-SNA of any one of claims 1-13, wherein each oligonucleotide in the shell of oligonucleotides is a targeting oligonucleotide.

15. The LNP-SNA of any one of claims 1-14, wherein at least one oligonucleotide in the shell of oligonucleotides comprises or consists of a (GGT)ₙ nucleotide sequence, wherein n is 2, 3, 4, 5, 6, 7, 8, 9, 10, 11, 12, 13, 14, 15, 16, 17, 18, 19, 20, or more.

16. The LNP-SNA of any one of claims 1-15, wherein each oligonucleotide in the shell of oligonucleotides comprises or consists of a (GGT)ₙ nucleotide sequence, wherein n is 2, 3, 4, 5, 6, 7, 8, 9, 10, 11, 12, 13, 14, 15, 16, 17, 18, 19, 20, or more.

17. The LNP-SNA of claim 15 or claim 16, wherein n is 7.

18. The LNP-SNA of any one of claims 1-17, wherein at least one oligonucleotide in the shell of oligonucleotides is an aptamer.

19. The LNP-SNA of any one of claims 1-18, wherein at least one oligonucleotide in the shell of oligonucleotides comprises a detectable marker.

20. The LNP-SNA of any one of claims 1-19, wherein the shell of oligonucleotides comprises an inhibitory oligonucleotide, an immunostimulatory oligonucleotide, a gene editor substrate DNA or RNA or a combination thereof.

21. The LNP-SNA of claim 20, wherein the inhibitory oligonucleotide is an antisense oligonucleotide, small interfering RNA (siRNA), an aptamer, a short hairpin RNA (shRNA), a DNAzyme, or an aptazyme.

22. The LNP-SNA of claim 20 or claim 21, wherein the immunostimulatory oligonucleotide is a CpG-motif containing oligonucleotide, a double-stranded DNA oligonucleotide, or a single-stranded RNA oligonucleotide.

23. The LNP-SNA of any one of claims 1-22, wherein the encapsulated oligonucleotide is comprised of DNA, RNA, or a combination thereof.

24. The LNP-SNA of any one of claims 1-23, wherein the encapsulated oligonucleotide is an inhibitory oligonucleotide, mRNA, an immunostimulatory oligonucleotide, a mRNA encoding a gene editor protein, or a DNA or RNA gene editor substrate.

25. The LNP-SNA of claim 24, wherein the inhibitory oligonucleotide is an antisense oligonucleotide, small interfering RNA (siRNA), an aptamer, a short hairpin RNA (shRNA), a DNAzyme, or an aptazyme.

26. The LNP-SNA of claim 24 or claim 25, wherein the immunostimulatory oligonucleotide is CpG-motif containing oligonucleotide.

27. The LNP-SNA of any one of claims 1-26, wherein the encapsulated oligonucleotide is about 5 to about 5000 nucleotides in length.

28. The LNP-SNA of any one of claims 1-27, wherein the encapsulated oligonucleotide is about 10 to about 4500 nucleotides in length.

29. The LNP-SNA of any one of claims 1-28, wherein the encapsulated oligonucleotide is about 1500 nucleotides in length.

30. The LNP-SNA of any one of claims 1-29, wherein the lipid nanoparticle core comprises a plurality of encapsulated oligonucleotides.

31. The LNP-SNA of claim 30, wherein at least one oligonucleotide in the plurality of encapsulated oligonucleotides comprises a detectable marker.

32. The LNP-SNA of claim 30 or claim 31, wherein the plurality of encapsulated oligonucleotides comprises an inhibitory oligonucleotide, mRNA, an immunostimulatory oligonucleotide, mRNA encoding a gene editor protein, a DNA or RNA gene editor substrate, or a combination thereof.

33. The LNP-SNA of claim 32, wherein the inhibitory oligonucleotide is an antisense oligonucleotide, small interfering RNA (siRNA), an aptamer, a short hairpin RNA (shRNA), a DNAzyme, or an aptazyme.

34. The LNP-SNA of claim 32 or claim 33, wherein the immunostimulatory oligonucleotide is a CpG-motif containing oligonucleotide, a double-stranded DNA (dsDNA), a double-stranded RNA, or a single-stranded RNA (ssRNA).

35. The LNP-SNA of any one of claims 30-32, wherein each oligonucleotide in the plurality of encapsulated oligonucleotides is about 10 to about 50 nucleotides in length.

36. The LNP-SNA of any one of claims 30-35, wherein each oligonucleotide in the plurality of encapsulated oligonucleotides is about 50 nucleotides in length.

37. The LNP-SNA of any one of claims 30-36, wherein each oligonucleotide in the plurality of encapsulated oligonucleotides has the same nucleotide sequence.

38. The LNP-SNA of any one of claims 30-36, wherein the plurality of encapsulated oligonucleotides comprises at least two oligonucleotides having different nucleotide sequences.

39. The LNP-SNA of any one of claims 1-38, wherein the ionizable lipid is dilinoleylmethyl-4-dimethylaminobutyrate (DLin-MC3-DMA), 2,2-Dilinoleyl-4-dimethylaminoethyl-[1,3]-dioxolane (DLin-KC2-DMA), C12-200, 1,2-dioleoyl-3-dimethylammonium-propane (DODAP), or a combination thereof.

40. The LNP-SNA of any one of claims 1-39, wherein the ionizable lipid is dilinoleylmethyl-4-dimethylaminobutyrate (DLin-MC3-DMA).

41. The LNP-SNA of any one of claims 1-40, wherein the LNP-SNA comprises a molar fraction of the ionizable lipid that is about 50% of the total lipid in the LNP-SNA.

42. The LNP-SNA of any one of claims 1-41, wherein the phospholipid is 1,2-distearoyl-sn-glycero-3-phosphocholine (DSPC), 1,2-Dihexadecanoyl phosphatidylcholine (DPPC), 1,2-dioleoyl-sn-glycero-3-phosphocholine (DOPC), 1,2-dioleoyl-sn-glycero-3-phosphoethanolamine (DOPE), or a combination thereof.

43. The LNP-SNA of any one of claims 1-42, wherein the phospholipid is 1,2-dioleoyl-sn-glycero-3-phosphoethanolamine (DOPE).

44. The LNP-SNA of any one of claims 1-43, wherein the LNP-SNA comprises a molar fraction of the phospholipid that is about 1% to about 25% of the total lipid in the LNP-SNA.

45. The LNP-SNA of any one of claims 1-44, wherein the LNP-SNA comprises a molar fraction of the phospholipid that is or is about 3.5% of the total lipid in the LNP-SNA.

46. The LNP-SNA of any one of claims 1-45, wherein the sterol is 3β-Hydroxycholest-5-ene (Cholesterol), 9,10-Secocholesta-5,7,10(19)-trien-3β-ol (Vitamin D3), 9,10-Secoergosta-5,7,10(19),22-tetraen-3β-ol (Vitamin D2), Calcipotriol, 24-Ethyl-5,22-cholestadien-3β-ol (Stigmasterol), 22,23-Dihydrostigmasterol (β-Sitosterol), 3,28-Dihydroxy-lupeol (Betulin), Lupeol, Ursolic acid, Oleanolic acid, 24α-Methylcholesterol (Campesterol), 24-Ethylcholesta-5,24(28)E-dien-3β-ol (Fucosterol), 24-Methylcholesta-5,22-dien-3β-ol (Brassicasterol), 24-Methylcholesta-5,7,22-trien-3β-ol (Ergosterol), 9,11-Dehydroergosterol, Daucosterol, or any of the foregoing sterols modified with one or more amino acids.

47. The LNP-SNA of any one of claims 1-46, wherein the LNP-SNA comprises a molar fraction of the sterol that is about 25% to about 45% of the total lipid in the LNP-SNA.

48. The LNP-SNA of any one of claims 1-47, wherein the LNP-SNA comprises a molar fraction of the sterol that is or is about 45% of the total lipid in the LNP-SNA.

49. The LNP-SNA of any one of claims 1-48, wherein the sterol is cholesterol.

50. The LNP-SNA of claim 49, wherein the LNP-SNA comprises a molar fraction of cholesterol that is or is about 45% of the total lipid in the LNP-SNA.

51. The LNP-SNA of any one of claims 1-50, wherein the lipid-polyethylene glycol (lipid-PEG) conjugate comprises 2000 Dalton (Da) polyethylene glycol.

52. The LNP-SNA of any one of claims 1-51, wherein the lipid-polyethylene glycol (lipid-PEG) conjugate is lipid-PEG-maleimide.

53. The LNP-SNA of claim 52, wherein the lipid-PEG-maleimide is 1,2-dipalmitoryl-sn-glycero-3-phosphoethanolamine (DPPE) conjugated to 2000 Da polyethylene glycol maleimide, 1,2-dimyristoyl-sn-glycero-3-phosphoethanolamine (DMPE) conjugated to 2000 Da polyethylene glycol maleimide, or a combination thereof.

54. The LNP-SNA of any one of claims 1-53, wherein the LNP-SNA comprises a molar fraction of the lipid-PEG conjugate that is about 1.5% to about 3.5% of the total lipid in the LNP-SNA.

55. The LNP-SNA of any one of claims 1-54, wherein the LNP-SNA comprises a molar fraction of the lipid-PEG conjugate that is or is about 1.5% of the total lipid in the LNP-SNA.

56. The LNP-SNA of any one of claims 1-55, wherein the mass ratio between the ionizable lipid and the encapsulated oligonucleotide is about 20:1 to about 5:1.

57. The LNP-SNA of any one of claims 1-56, further comprising a therapeutic agent encapsulated in the lipid nanoparticle core.

58. The LNP-SNA of any one of claims 1-57, further comprising a therapeutic agent attached to the exterior of the lipid nanoparticle core.

59. The LNP-SNA of claim 57 or claim 58, wherein the therapeutic agent is an antibody or antibody fragment, a small molecule, a peptide, an antibiotic, a growth factor, a hormone, an interferon, an interleukin, an antifungal, an antiviral, a chemotherapeutic agent, or a combination thereof.

60. The LNP-SNA of any one of claims 1-59, further comprising a targeting peptide, targeting antibody, or a combination thereof attached to the exterior of the lipid nanoparticle core.

61. A composition comprising a plurality of the lipid nanoparticle spherical nucleic acids (LNP-SNAs) of any one of claims 1-60.

62. The composition of claim 61, further comprising a therapeutic agent.

63. A method of inhibiting expression of a gene comprising the step of hybridizing a polynucleotide encoding the gene product with the lipid nanoparticle spherical nucleic acid (LNP-SNA) of any one of claims 1-60, or the composition of claim 61 or claim 62, wherein hybridizing between the polynucleotide and one or more oligonucleotides in the shell of oligonucleotides occurs over a length of the polynucleotide with a degree of complementarity sufficient to inhibit expression of the gene product.

64. The method of claim 63 wherein expression of the gene product is inhibited in vivo.

65. The method of claim 63 wherein expression of the gene product is inhibited in vitro.

66. A method for up-regulating activity of a toll-like receptor (TLR), comprising contacting a cell having the toll-like receptor with the lipid nanoparticle spherical nucleic acid (LNP-SNA) of any one of claims 1-60, or the composition of claim 61 or claim 62.

67. The method of claim 66 wherein the shell of oligonucleotide comprises one or more oligonucleotides that is a TLR agonist.

68. The method of claim 66 or claim 67 wherein the toll-like receptor is toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, toll-like receptor 13, or a combination thereof.

69. A method for down-regulating activity of a toll-like receptor (TLR), comprising contacting a cell having the toll-like receptor with the lipid nanoparticle spherical nucleic acid (LNP-SNA) of any one of claims 1-60, or the composition of claim 61 or claim 62.

70. The method of claim 69 wherein the shell of oligonucleotides comprises one or more oligonucleotides that is a TLR antagonist.

71. The method of claim 69 or claim 70 wherein the toll-like receptor is toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, toll-like receptor 13, or a combination thereof.

72. The method of any one of claims 66-71 which is performed in vitro.

73. The method of any one of claims 66-71 which is performed in vivo.

74. A method of treating a disorder comprising administering an effective amount of the lipid nanoparticle spherical nucleic acid (LNP-SNA) of any one of claims 1-60 or the composition of claim 61 or claim 62 to a subject in need thereof, wherein the administering treats the disorder.

75. The method of claim 74, wherein the disorder is cancer, an infectious disease, an autoimmune disease, or a combination thereof.
